SEQUENCE LISTING (36 sequences)

SEQ ID NO: 1 - DNA, 30 bp, Artificial sequence
  Synthetic polynucleotide
  misc_feature (1)..(3): Residue modified at 3′-end with phosphorothioate bonded to 5′-end of neighboring residue
  misc_feature (28)..(30): Residue modified at 5′-end with phosphorothioate bonded to 3′-end of neighboring residue
aaaaaaaaaaaaaaaaaaaaaaaaaaaaaa

SEQ ID NO: 2 - DNA, 30 bp, Artificial sequence
  Synthetic polynucleotide
  misc_feature (1)..(3): Residue modified at 3′-end with phosphorothioate bonded to 5′-end of neighboring residue
  misc_feature (28)..(30): Residue modified at 5′-end with phosphorothioate bonded to 3′-end of neighboring residue
tttttttttttttttttttttttttttttt

SEQ ID NO: 3 - DNA, 50 bp, Artificial sequence
  Synthetic polynucleotide
  misc_feature (1)..(3): Residue modified at 3′-end with thosphorothioate bonded to 5′-end of neighboring residue
  misc_feature (48)..(50): Residue modified at 5′-end with phosphorothioate bonded to 3′-end of neighboring residue
aaaaaaaaaaaaaaaaaaaaaaaaaaaaaaaaaaaaaaaaaaaaaaaaaa

SEQ ID NO: 4 - DNA, 50 bp, Artificial sequence
  Synthetic polynucleotide
  misc_feature (1)..(3): Residue modified at 3′-end with phosphorothioate bonded to 5′-end of neighboring residue
  misc_feature (48)..(50): Residue modified at 5′-end with phosphorothioate attached to 3′-end of neighboring residue
  misc_feature (48)..(50): Residue modified at 5′-end with phosphorothioate bonded to 3′-end of neighboring residue
tttttttttttttttttttttttttttttttttttttttttttttttttt

SEQ ID NO: 5 - DNA, 60 bp, Artificial sequence
  Synthetic polynucleotide
  misc_feature (1)..(3): Residue modified at 3′-end with phosphorothioate bonded to 5′-end of neighboring residue
  misc_feature (58)..(60): Residue modified at 5′-end with phosphorothioate bonded to 3′-end of neighboring residue
aaaaaaaaaaaaaaaaaaaaaaaaaaaaaaaaaaaaaaaaaaaaaaaaaaaaaaaaaaaa

SEQ ID NO: 6 - DNA, 60 bp, Artificial sequence
  Synthetic polynucleotide
  misc_feature (1)..(3): Residue modified at 3′-end with phosphorothioate bonded to 5′-end of neighboring residue
  misc_feature (58)..(60): Residue modified at 5′-end with phosphorothioate attached to 3′-end of neighboring residue
  misc_feature (58)..(60): Residue modified at 5′-end with phosphorothioate bonded to 3′-end of neighboring residue
tttttttttttttttttttttttttttttttttttttttttttttttttttttttttttt

SEQ ID NO: 7 - DNA, 70 bp, Artificial sequence
  Synthetic polynucleotide
  misc_feature (1)..(3): Residue modified at 3′-end with phosphorothioate bonded to 5′-end of neighboring residue
  misc_feature (68)..(70): Residue modified at 5′-end with phosphorothioate bonded to 3′-end of neighboring residue
aaaaaaaaaaaaaaaaaaaaaaaaaaaaaaaaaaaaaaaaaaaaaaaaaaaaaaaaaaaaaaaaaaaaaa

SEQ ID NO: 8 - DNA, 70 bp, Artificial sequence
  Synthetic polynucleotide
  misc_feature (1)..(3): Residue modified at 3′-end with phosphorothioate bonded to 5′-end of neighboring residue
  misc_feature (68)..(70): Residue modified at 5′-end with phosphorothioate bonded to 3′-end of neighboring residue
tttttttttttttttttttttttttttttttttttttttttttttttttttttttttttttttttttttt

SEQ ID NO: 9 - DNA, 80 bp, Artificial sequence
  Synthetic polynucleotide
  misc_feature (1)..(3): Residue modified at 3′-end with phosphorothioate bonded to 5′-end of neighboring residue
  misc_feature (78)..(80): Residue modified at 5′-end with phosphorothioate bonded to 3′-end of neighboring residue
aaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - DNA, 80 bp, Artificial sequence
  Synthetic polynucleotide
  misc_feature (1)..(3): Residue modified at 3′-end with phosphorothioate bonded to 5′-end of neighboring residue
  misc_feature (78)..(80): Residue modified at 5′-end with phosphorothioate bonded to 3′-end of neighboring residue
tttttttttttttttttttttttttttttttttttttttttttttttttttttttttttttttttttttttttttttttt

SEQ ID NO: 11 - DNA, 90 bp, Artificial sequence
  Synthetic polynucleotide
  misc_feature (1)..(3): Residue modified at 3′-end with phosphorothioate bonded to 5′-end of neighboring residue
  misc_feature (88)..(90): Residue modified at 5′-end with phosphorothioate bonded to 3′-end of neighboring residue
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 90 bp, Artificial sequence
  Synthetic polynucleotide
  misc_feature (1)..(3): Residue modified at 3′-end with phosphorothioate bonded to 5′-end of neighboring residue
  misc_feature (88)..(90): Residue modified at 5′-end with phosphorothioate bonded to 3′-end of neighboring residue
tttttttttttttttttttttttttttttttttttttttttttttttttttttttttttttttttttttttttttttttttttttttttt

SEQ ID NO: 13 - DNA, 100 bp, Artificial sequence
  Synthetic polynucleotide
  misc_feature (1)..(3): Residue modified at 3′-end with phosphorothioate bonded to 5′-end of neighboring residue
  misc_feature (98)..(100): Residue modified at 5′-end with phosphorothioate bonded to 3′-end of neighboring residue
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - DNA, 100 bp, Artificial sequence
  Synthetic polynucleotide
  misc_feature (1)..(3): Residue modified at 3′-end with phosphorothioate bonded to 5′-end of neighboring residue
  misc_feature (98)..(100): Residue modified at 5′-end with phosphorothioate bonded to 3′-end of neighboring residue
tttttttttttttttttttttttttttttttttttttttttttttttttttttttttttttttttttttttttttttttttttttttttttttttttttt

SEQ ID NO: 15 - DNA, 110 bp, Artificial sequence
  Synthetic polynucleotide
  misc_feature (1)..(3): Residue modified at 3′-end with phosphorothioate bonded to 5′-end of neighboring residue
  misc_feature (108)..(110): Residue modified at 5′-end with phosphorothioate bonded to 3′-end of neighboring residue
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - DNA, 110 bp, Artificial sequence
  Synthetic polynucleotide
  misc_feature (1)..(3): Residue modified at 3′-end with phosphorothioate bonded to 5′-end of neighboring residue
  misc_feature (108)..(110): Residue modified at 5′-end with phosphorothioate bonded to 3′-end of neighboring residue
tttttttttttttttttttttttttttttttttttttttttttttttttttttttttttttttttttttttttttttttttttttttttttttttttttttttttttttt

SEQ ID NO: 17 - DNA, 30 bp, Artificial sequence
  Synthetic polynucleotide
  misc_feature (1)..(3): Residue modified at 3′-end with phosphorothioate bonded to 5′-end of neighboring residue
  misc_feature (28)..(30): Residue modified at 5′-end with phosphorothioate bonded to 3′-end of neighboring residue
gcgcgcgcgcgcgcgcgcgcgcgcgcgcgc

SEQ ID NO: 18 - DNA, 50 bp, Artificial sequence
  Synthetic polynucleotide
  misc_feature (1)..(3): Residue modified at 3′-end with phosphorothioate bonded to 5′-end of neighboring residue
  misc_feature (48)..(50): Residue modified at 5′-end with phosphorothioate bonded to 3′-end of neighboring residue
gcgcgcgcgcgcgcgcgcgcgcgcgcgcgcgcgcgcgcgcgcgcgcgcgc

SEQ ID NO: 19 - DNA, 60 bp, Artificial sequence
  Synthetic polynucleotide
  misc_feature (1)..(3): Residue modified at 3′-end with phosphorothioate bonded to 5′-end of neighboring residue
  misc_feature (58)..(60): Residue modified at 5′-end with phosphorothioate bonded to 3′-end of neighboring residue
gcgcgcgcgcgcgcgcgcgcgcgcgcgcgcgcgcgcgcgcgcgcgcgcgcgcgcgcgcgc

SEQ ID NO: 20 - DNA, 70 bp, Artificial sequence
  Synthetic polynucleotide
  misc_feature (1)..(3): Residue modified at 3′-end with phosphorothioate bonded to 5′-end of neighboring residue
  misc_feature (68)..(70): Residue modified at 5′-end with phosphorothioate bonded to 3′-end of neighboring residue
gcgcgcgcgcgcgcgcgcgcgcgcgcgcgcgcgcgcgcgcgcgcgcgcgcgcgcgcgcgcgcgcgcgcgc

SEQ ID NO: 21 - DNA, 80 bp, Artificial sequence
  Synthetic polynucleotide
  misc_feature (1)..(3): Residue modified at 3′-end with phosphorothioate bonded to 5′-end of neighboring residue
  misc_feature (78)..(80): Residue modified at 3′-end with phosphorothioate bonded to 5′-end of neighboring residue
gcgcgcgcgcgcgcgcgcgcgcgcgcgcgcgcgcgcgcgcgcgcgcgcgcgcgcgcgcgcgcgcgcgcgcgcgcgcgcgc

SEQ ID NO: 22 - DNA, 90 bp, Artificial sequence
  Synthetic polynucleotide
  misc_feature (1)..(3): Residue modified at 3′-end with phosphorothioate bonded to 5′-end of neighboring residue
  misc_feature (88)..(90): Residue modified at 5′-end with phosphorothioate bonded to 3′-end of neighboring residue
gcgcgcgcgcgcgcgcgcgcgcgcgcgcgcgcgcgcgcgcgcgcgcgcgcgcgcgcgcgcgcgcgcgcgcgcgcgcgcgcgcgcgcgcgc

SEQ ID NO: 23 - DNA, 100 bp, Artificial sequence
  Synthetic polynucleotide
  misc_feature (1)..(3): Residue modified at 3′-end with phosphorothioate bonded to 5′-end of neighboring residue
  misc_feature (09)..(100): Residue modified at 5′-end with phosphorothioate bonded to 3′-end of neighboring residue
gcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgc

SEQ ID NO: 24 - DNA, 76 bp, Artificial sequence
  Synthetic polynucleotide
  misc_feature (1)..(3): Residue modified at 3′-end with phosphorothioate bonded to 5′-end of neighboring residue
  misc_feature (74)..(76): Residue modified at 5′-end with phosphorothioate bonded to 3′-end of neighboring residue
aaaaaaaaaaaaaaaaaaaaaaaaaaaaaaaaaaaaaaaaaaaaaaaaaaaaaaaaaaaaaaaaaaaaaaaaaaaa

SEQ ID NO: 25 - DNA, 76 bp, Artificial sequence
  Synthetic polynucleotide
  misc_feature (1)..(3): Residue modified at 3′-end with phosphorothioate bonded to 5′-end of neighboring residue
  misc_feature (74)..(76): Residue modified at 5′-end with phosphorothioate bonded to 3′-end of neighboring residue
tttttttttttttttttttttttttttttttttttttttttttttttttttttttttttttttttttttttttttt

SEQ ID NO: 26 - DNA, 76 bp, Artificial sequence
  Synthetic polynucleotide
  misc_feature (1)..(3): Residue modified at 3′-end with phosphorothioate bonded to 5′-end of neighboring residue
  misc_feature (74)..(76): Residue modified at 5′-end with phosphorothioate bonded to 3′-end of neighboring residue
acacacacacacacacacacacacacacacacacacacacacacacacacacacacacacacacacacacacacac

SEQ ID NO: 27 - DNA, 76 bp, Artificial sequence
  Synthetic polynucleotide
  misc_feature (1)..(3): Residue modified at 3′-end with phosphorothioate bonded to 5′-end of neighboring residue
  misc_feature (74)..(76): Residue modified at 5′-end with phosphorothioate bonded to 3′-end of neighboring residue
gtgtgtgtgtgtgtgtgtgtgtgtgtgtgtgtgtgtgtgtgtgtgtgtgtgtgtgtgtgtgtgtgtgtgtgtgtgt

SEQ ID NO: 28 - DNA, 76 bp, Artificial sequence
  Synthetic polynucleotide
  misc_feature (1)..(3): Residue modified at 3′-end with phosphorothioate bonded to 5′-end of neighboring residue
  misc_feature (74)..(76): Residue modified at 5′-end with phosphorothioate bonded to 3′-end of neighboring residue
atatatatatatatatatatatatatatatatatatatatatatatatatatatatatatatatatatatatatat

SEQ ID NO: 29 - DNA, 76 bp, Artificial sequence
  Synthetic polynucleotide
  misc_feature (1)..(3): Residue modified at 3′-end with phosphorothioate bonded to 5′-end of neighboring residue
  misc_feature (74)..(76): Residue modified at 5′-end with phosphorothioate bonded to 3′-end of neighboring residue
tatatatatatatatatatatatatatatatatatatatatatatatatatatatatatatatatatatatatata

SEQ ID NO: 30 - DNA, 76 bp, Artificial sequence
  Synthetic polynucleotide
  misc_feature (1)..(3): Residue modified at 3′-end with phosphorothioate bonded to 5′-end of neighboring residue
  misc_feature (74)..(76): Residue modified at 5′-end with phosphorothioate bonded to 3′-end of neighboring residue
actgactgactgactgactgactgactgactgactgactgactgactgactgactgactgactgactgactgactg

SEQ ID NO: 31 - DNA, 76 bp, Artificial sequence
  Synthetic polynucleotide
  misc_feature (1)..(3): Residue modified at 3′-end with phosphorothioate bonded to 5′-end of neighboring residue
  misc_feature (74)..(76): Residue modified at 5′-end with phosphorothioate bonded to 3′-end of neighboring residue
cagtcagtcagtcagtcagtcagtcagtcagtcagtcagtcagtcagtcagtcagtcagtcagtcagtcagtcagt

SEQ ID NO: 32 - DNA, 76 bp, Artificial sequence
  Synthetic polynucleotide
  misc_feature (1)..(3): Residue modified at 3′-end with phosphorothioate bonded to 5′-end of neighboring residue
  misc_feature (74)..(76): Residue modified at 5′-end with phosphorothioate bonded to 3′-end of neighboring residue
gaccctatcgatacagggcacggggtcgaactgttgggtttcgccatggtaccccctgcatttatatagccagacc

SEQ ID NO: 33 - DNA, 76 bp, Artificial sequence
  Synthetic polynucleotide
  misc_feature (1)..(3): Residue modified at 3′-end with phosphorothioate bonded to 5′-end of neighboring residue
  misc_feature (74)..(76): Residue modified at 5′-end with phosphorothioate bonded to 3′-end of neighboring residue
ggtctggctatataaatgcagggggtaccatggcgaaacccaacagttcgaccccgtgccctgtatcgatagggtc

SEQ ID NO: 34 - protein, 8 aa, Artificial sequence
  Synthetic polypeptide
SIINFEKL

SEQ ID NO: 35 - DNA, 90 bp, Artificial sequence; identical to SEQ ID NO: 11
  Synthetic polynucleotide
  misc_feature (1)..(1): Residue modified with FAM (5(6)-carboxyfluorescein)
  misc_feature (1)..(3): Residue modified at 3′-end with phosphorothioate bonded to 5′-end of neighboring residue
  misc_feature (88)..(90): Residue modified at 5′-end with phosphorothioate bonded to 3′-end of neighboring residue

SEQ ID NO: 36 - DNA, 90 bp, Artificial sequence; identical to SEQ ID NO: 12
  Synthetic polynucleotide
  misc_feature (1)..(1): Residue modified with FAM (5(6)-carboxyfluorescein)
  misc_feature (1)..(3): Residue modified at 3′-end with phosphorothioate bonded to 5′-end of neighboring residue
  misc_feature (88)..(90): Residue modified at 5′-end with phosphorothioate bonded to 3′-end of neighboring residue

(19) United States
(12) Patent Application Publication
(10) Pub. No.: US 2021/0324023 A1
(43) Pub. Date: Oct. 21, 2021

(54) MODULATING IMMUNE RESPONSES

(71) Applicant: Glen N. Barber, Miami, FL (US)

(72) Inventor: Glen N. Barber, Miami, FL (US)

(21) Appl. No.: 17/357,822

(22) Filed: Jun. 24, 2021

Related U.S. Application Data
(63) Continuation-in-part of application No. 16/717,325, filed on Dec. 17, 2019.
Application No. 16/717,325 is a continuation of application No. 15/120,694, filed on Aug. 22, 2016, now abandoned.
Application No. 15/120,694 is a continuation-in-part of application No. 13/057,662, filed on Jun. 14, 2011, now abandoned.
(60) Provisional application No. 61/129,975, filed on Aug. 4, 2008.

(51) Int. Cl. C07K 14/47; C12N 15/113; A61K 31/136; A61K 31/196; A61K 31/4748; A61K 31/517; A61K 31/7048; A61K 31/711; A61K 31/713; A61K 38/17; A61K 45/06

(52) U.S. Cl. CPC C07K 14/4702; A61K 2300/00; A61K 31/136; A61K 31/196; A61K 31/4748; A61K 31/517; A61K 31/7048; A61K 31/711; A61K 31/713; A61K 38/1709; A61K 45/06; C12N 15/113; Y10T 436/143333; C12N 2310/14; C12N 2310/11; C12N 15/1138

(57) ABSTRACT

Modulators of STING are able to upregulate or down regulate immune responses. Administration of such modulators can be used to treat diseases or other undesirable conditions in a subject either directly or in combination with other agents.

### PRIORITY CLAIM

This application is a continuation-in-part of (1) U.S. application Ser. No. 16/717,325, filed Dec. 17, 2019,which is a continuation of (2) U.S. application Ser. No. 15/120,694, filed Aug. 22, 2016, which in turn is the national phase of (3) International Application PCT/US13/038840, filed Apr. 30, 2013, which is a continuation-in-part of (4) U.S. application Ser. No. 13/460,408, filed Apr. 30, 2012, which is a continuation-in-part of (5) U.S. application Ser. No. 13/057,662, filed Jun. 14, 2011, which is the national phase of (6) International Application PCT/US09/052767, filed Aug. 4, 2009, which claims the benefit of priority to (7) U.S. Provisional Application No. 61/129,975 filed Aug. 4, 2008, which applications (1)-(7) are hereby expressly incorporated by reference in their entireties for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file STNG-01000US4_ST25.TXT, created Jun. 18, 2021, 20,906 bytes, machine format IBM-PC, MS-Windows operating system, is hereby incorporated by references in its entirety and for all purposes.

### GOVERNMENT RIGHTS

The invention described herein was made with U.S. government support under grant number R01A1079336 awarded by the National Institutes of Health. The U.S. government has certain rights in the invention.

### FIELD OF THE INVENTION

Embodiments of the invention relate to compositions and methods for modulating innate and adaptive immunity in a subject and/or for the treatment of an immune-related disorder, cancer, autoimmunity, treating and preventing infections.

### BACKGROUND OF THE INVENTION

Cellular host defense responses to pathogen invasion principally involves the detection of pathogen associated molecular patterns (PAMPs) such as viral nucleic acid or bacterial cell wall components including lipopolysaccharide or flagellar proteins that results in the induction of anti-pathogen genes. For example, viral Ribonucleic Acid (RNA) can be detected by membrane bound Toll-like receptors (TLR's) present in the Endoplasmic Reticulum (ER) and/or endosomes (e.g. Toll-like receptor 3 (TLR 3) and TLR7/TLR8) or by TLR-independent intracellular DExD/H box RNA helicases referred to as Retinoic acid Inducible Gene 1 (RIG-1) or Melanoma Differentiation associated Antigen 5 (MDA5), also referred to as IFIH1 and helicard. These events culminate in the activation of downstream signaling events, much of which remains unknown, leading to the transcription of Nuclear Factor kappa-light-chain-enhancer of activated B cells (NF-KB) and Interferon Regulatory Factor 3 (IRF3)/IRF7-dependent genes, including type I Interferon (IFN).

### SUMMARY OF THE INVENTION

Tumor cells are notoriously non-immunogenic through their ability to mimic the properties of normal cells which have naturally evolved to avoid activating the immune system following cell death and phagocytosis. In an embodiment of the present invention, a new approach overcomes this obstacle and makes previously immuno-evasive, inert tumor cells highly immunogenic. This has been achieved through developing activators of the Stimulator of Interferon Genes (STING)-dependent innate immune signaling pathway, a strategy which holds considerable promise for the therapeutic treatment of cancer.

STING, a molecule that plays a key role in the innate immune response, includes 5 putative transmembrane (TM) regions, predominantly resides in the endoplasmic reticulum (ER), and is able to activate both NF-κβ and IRF3 transcription pathways to induce type I IFN and to exert a potent anti-viral state following expression (see U.S. patent application Ser. No. 16/717,325 and PCT/US2009/052767 each of which is incorporated herein by reference in its entirety and for all purposes). Loss of STING reduced the ability of Polyinosinic:polycytidylic acid (polyIC) to activate type I IFN and rendered Murine Embryonic Fibroblasts (MEFs) lacking STING (^{-/-} MEFs) generated by targeted homologous recombination, susceptible to vesicular stomatitis virus (VSV) infection. In the absence of STING, DNA-mediated type I IFN responses were inhibited, indicating that STING may play an important role in recognizing DNA from viruses, bacteria, and other pathogens which can infect cells. Yeast-two hybrid and co-immunoprecipitation studies indicated that STING interacts with RIG-1 and with Ssr2/TRAPβ, a member of the translocon-associated protein (TRAP) complex required for protein translocation across the ER membrane following translation. RNAi ablation of TRAPβ inhibited STING function and impeded the production of type I IFN in response to polyIC.

Further experiments showed that STING itself binds nucleic acids including single- and double-stranded DNA such as from pathogens and apoptotic DNA, and plays a central role in regulating pro-inflammatory gene expression in inflammatory conditions such as DNA-mediated arthritis and cancer. Various new methods of, and compositions for, upregulating STING expression or function are described herein along with further characterization of other cellular molecule which interact with STING. These discoveries allow for the design of new adjuvants, vaccines and therapies to regulate the immune system and other systems.

Described herein are methods for modulating an immune response in a subject having a disease or disorder associated with aberrant STING function. These methods can include the step of administering to the subject an amount of a pharmaceutical composition including an agent which modulates STING function and a pharmaceutically acceptable carrier, wherein amount the pharmaceutical composition is effective to ameliorate the aberrant STING function in the subject. The agent can be a small molecule that increases or decreases STING function, or a nucleic acid molecule that binds to STING under intracellular conditions. The STING-binding nucleic acid molecule can be a single-stranded DNA between 40 and 150 base pairs in length or a double-stranded DNA between 40 and 150, 60 and 120, 80 and 100, or 85 and 95 base pairs in length or longer. The STING-binding nucleic acid molecule can be nuclease-resistant, e.g., made up of nuclease-resistant nucleotides. It can also be associated with a molecule that facilitates transmembrane transport. In these methods, the disease or disorder can be a DNA-dependent inflammatory disease.

Also described herein are methods of treating cancer in a subject having a cancerous tumor infiltrated with inflammatory immune cells. These methods can include the step of administering to the subject an amount of a pharmaceutical composition including an agent which downregulates STING function or expression and a pharmaceutically acceptable carrier, wherein amount the pharmaceutical composition is effective to reduce the number of inflammatory immune cells infiltrating the cancerous tumor by at least 50% (e.g., at least 50, 60, 70, 80, or 90%, or until reduction of inflammatory cell infiltration is detectably reduced by histology or scanning).

In an embodiment of the present invention, autologous tumor cells loaded with STING-dependent adjuvants (STAVs) can be reinfused into a patient to stimulate Antigen Presenting Cells (APCs) in vivo and thus anti-tumor Cytotoxic T cells (CTLs). STAV loaded cells are highly immunogenic, and therefore potent activators of APC's. In various embodiments of the present invention, the strategy is applicable to patients suffering from highly aggressive leukemia, specifically relapsed/refractory Acute Myeloid Leukemia (AML) and Adult Lymphocytic Leukemia (ALL) such as HTLV-1 associated Adult T cell Lymphocytic Leukemia (ATLL). In various alternative embodiments of the present invention, the strategy is applicable to a variety of cancers, not just leukemia.

The details of the disclosure are set forth in the accompanying description below. Although methods and materials similar or equivalent to those described herein can be used in the practice or testing of the present application, illustrative methods and materials are now described. In the case of conflict, the present specification, including definitions, will control. In addition, the materials, methods, and examples are illustrative only and are not intended to be limiting. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### BRIEF DESCRIPTION OF THE DRAWINGS

Various embodiments of the present invention will be described in detail based on the following Figures, where:

FIG. 1A is a histogram showing an IFNβ ELISA assay in mouse embryonic fibroblasts (MEFs) Wild Type (WT) or STING Knock Out (SKO) cells transfected with different lengths of AT rich-STING ligands (lipofectamine 2000 transfection reagent only (11); A:T30ES (22) (SEQ ID NO:1, SEQ ID NO:2); A:T50ES (23) (SEQ ID NO:3, SEQ ID NO:4); A:T60ES (24) (SEQ ID NO:5, SEQ ID NO:6); A:T70ES (25) (SEQ ID NO:7, SEQ ID NO:8); A:T80ES (26) (SEQ ID NO:9, SEQ ID NO:10); A:T90ES (27) (SEQ ID NO:11, SEQ ID NO:12); and A:T100ES (28) (SEQ ID NO:13, SEQ ID NO:14));

FIG. 1B is a histogram showing an IFNβ ELISA assay in hTERT fibroblast transfected with different lengths of AT rich-STING ligands ((11); A:T30ES (22), A:T50ES (23), A:T60ES (24); A:T70ES (25); A:T80ES (26); A:T90ES (27); and A:T100ES (28));

FIG. 1C is a histogram showing a quantitative Real Time—Polymerase Chain Reaction (qRT-PCR) analysis of IFNβ1 in human macrophages transfected with different length of AT rich-STING ligands ((11); A:T30ES (22); A:T50ES (23); A:T60ES (24); A:T70ES (25); A:T80ES (26); A:T90ES (27); A:T100ES (28); and A:T110ES (29) (SEQ ID NO:15, SEQ ID NO:16));

FIG. 1D is a histogram showing an IFNβ ELISA assay in MEFs WT or SKO cells transfected with different lengths of GC rich-STING ligands ((11); GC30ES (32) (SEQ ID NO:17); GC50ES (33) (SEQ ID No:18); GC60ES (34) (SEQ ID NO:19); GC70ES (35) (SEQ ID NO:20); GC80ES (36) (SEQ ID NO:21); GC90ES (37) (SEQ ID NO:22); and GC100ES (38) (SEQ ID NO:23));

FIG. 1E is a histogram showing an IFNβ ELISA assay hTERT fibroblast transfected with different lengths of GC rich-STING ligands ((11); GC30ES (32); GC50ES (33); GC60ES (34); GC70ES (35); GC80ES (36); GC90ES (37); and GC100ES (38));

FIG. 1F is a histogram showing a qRT-PCR analysis of IFNβ1 in human macrophages transfected with different length of GC rich-STING ligands ((11); GC30ES (32); GC50ES (33); GC60ES (34); GC70ES (35); GC80ES (36); GC90ES (37); and GC100ES (38));

FIG. 2A shows a schematic representation of intratumoral injection of STAVs in B16 OVA melanoma bearing mice (the mice were subcutaneously (s.c.) injected with B16-OVA cells on the flank, where 10 μg of STAVs were injected intratumorally (i.t.) every 3 days);

FIG. 2B shows the growth in tumor volumes from WT (n=7/group) mice s.c. injected with murine B16 melanoma cells (B16-OVA cells) on the flank and subsequently injected i.t. every 3 days with 10 μg of STAVs (STAV 1, Poly A:T76 ES, SEQ ID NO:24, SEQ ID NO:25; STAV 2, Poly AC:TG76 ES, SEQ ID NO:26, SEQ ID NO:27; STAV 3, Poly AT76 ES, SEQ ID NO:28, SEQ ID NO:29; STAV 4, ACTG76 ES, SEQ ID NO:30, SEQ ID NO:31; STAV 5, HSV RL2 intron, HSV RL2 intron-S (SEQ ID NO:32), HSV RL2 intron-AS (SEQ ID NO:33)) or Phosphate Buffered Saline (PBS) (as a control) and measured on the indicated days;

FIG. 2C shows the growth in tumor volumes from SKO (n=7/group) mice s.c. injected with murine B16 melanoma cells (B16-OVA cells) on the flank and subsequently injected i.t. every 3 days with 10 μg of STAVs or PBS (as control) and measured on the indicated days;

FIG. 2D is a histogram showing the frequency of OVA specific CD8+ T cells (using OVA257-264 (SIINFEKL) peptide, SEQ ID NO:34) in the spleen from: WT (n=4/group) mice injected with PBS as control (12), with STAV (44); SKO (n=4/group) mice injected with PBS as control (13), with STAV (45);

FIG. 3A shows confocal microscope analyses of B16 OVA cells transfected with no DNA (14), STAV-FAM (FAM labeled STAVs) (52) (SEQ ID NO:35, SEQ ID NO:36) with DAPI, anti-calreticulin counter staining;

FIG. 3B shows Fluorescence-Activated Cell Sorting (FACS) analyses of B16 OVA cells transfected with no DNA (14), FAM labeled STAVs (52), bar size, 10 μm;

FIG. 3C shows a schematic representation of the phagocytosis of B16 cells by macrophages (B16 cells were transfected by 3 μg/ml of STAVs for 3 hours and ultraviolet (UV) irradiated (120 mJ/cm), the irradiated B16 cells were fed to macrophages at 24 hours after UV irradiation);

FIG. 3D shows confocal microscope analyses in macrophages transfected with lipo only (11) following cellular engulfment of B16 cells transfected with no DNA (14) or FAM labeled STAVs (52), data are representative of at least 3 independent experiments;

FIG. 3E is a histogram showing FACS analyses in macrophages following cellular engulfment of B16 cells transfected with no DNA (14) or FAM labeled STAVs (52) analyzed at 4, 24 and 48 hours, data are representative of at least 3 independent experiments;

FIG. 3F is a histogram showing qRT-PCR analysis of IFNβ1 in WT macrophages (15) and SKO macrophages (53): Mock (11) and following engulfment of B16 cells in absence of STAVs (14) or presence of STAVs (52), data are representative of at least 3 independent experiments, error bars indicate mean±SD, *, p<0.05; Student's t-test;

FIG. 3G is a histogram showing FACS analysis for CD86 in CD11b+ WT macrophages (15) and CD11b+ SKO macrophages (53): Mock (11) and following engulfment of B16 cells in absence of STAVs (14) or presence of STAVs (52), data are representative of at least 3 independent experiments;

FIG. 3H is a histogram showing FACS analysis for CD86 in CD8a+CD11C+ WT dendritic cells (15) and SKO dendritic cells (53): Mock (11) and following engulfment of B16 cells in absence of STAVs (14) or presence of STAVs (52), data are representative of at least 3 independent experiments;

FIG. 4A shows a schematic representation of the dead cell immunization protocol where B16 OVA cells were transfected with STAVs for 3 hours, then UV irradiated (120 mJ/cm), incubated for 24 hours and then the cells were i.p. injected into mice;

FIG. 4B shows an IFNγ measurement in splenocytes where B16 OVA cells were transfected with STAVs for 3 hours, then UV irradiated (120 mJ/cm), incubated for 24 hours and then the dead cells were i.p. injected into WT (16), SKO (54), cGAS KO (55), and TLR9 KO (56) mice at 7 days after the second immunization, error bars indicate mean±SD;

FIG. 4C shows a schematic representation of post-vaccination protocol for B16 OVA mediated lung metastasis in mice;

FIG. 4D is a histogram showing post-vaccination survival rates for B16 OVA mediated lung metastasis from WT mice (16) i.v. injected with B16 OVA cells (5×104 cells/mouse), on days 1, 3, 7, and 14, mice were i.p. injected with UV irradiated B16 OVA cells (1×106 cells/mouse) with STAVs (p=0.0429, n=7/group, p values were based on Log-rank test, with p<0.05 considered statistically significant), where PBS treated (11) and B16 with no DNA transfected treated (14) are also shown;

FIG. 4E is a histogram showing post-vaccination survival rates for B16 OVA mediated lung metastasis from cGAS KO mice (55) i.v. injected with B16 OVA cells (5×104 cells/mouse), on days 1, 3, 7, and 14, mice were i.p. injected with UV irradiated B16 OVA cells (1×10 cells/mouse) with STAVs (p=0.4075, n=7/group, p values were based on Log-rank test, with p<0.05 considered statistically significant), where PBS treated (11) and B16 with no DNA transfected treated (14) are also shown;

FIG. 4F is a histogram showing post-vaccination survival rates for B16 OVA mediated lung metastasis from TLR9 KO mice (56) i.v. injected with B16 OVA cells (5×104 cells/mouse), on days 1, 3, 7, and 14, mice were i.p. injected with UV irradiated B16 OVA cells (1×106 cells/mouse) with STAVs (p=0.0012, n=7/group, p values were based on Log-rank test, with p<0.05 considered statistically significant), where PBS treated (11) and B16 with no DNA transfected treated (14) are also shown;

FIG. 4G is a histogram showing post-vaccination survival rates for B16 OVA mediated lung metastasis from SKO mice (54) i.v. injected with B16 OVA cells (5×104 cells/mouse), on days 1, 3, 7, and 14, mice were i.p. injected with UV irradiated B16 OVA cells (1×106 cells/mouse) with STAVs (p=0.2616, n=7/group, p values were based on Log-rank test, with p<0.05 considered statistically significant), where PBS treated (11) and B16 with no DNA transfected treated (14) are also shown;

FIG. 5A shows a schematic representation of the phagocytosis of EL4-HBZ cells by Bone Marrow Derived Macrophages (BMDM) from WT or SKO mice; EL4-HBZ cells (1×10⁶) were transfected with STAVs (3 ug/ml) for 3 hours and irradiated by UV (120 mJ/cm) (at 24 hours after UV irradiation, the irradiated EL4-HBZ cells were fed to WT macrophages (WT BMDM) or SKO macrophages (SKO BMDM));

FIG. 5B shows qPCR analysis of IFNβ in WT BMDM (16), WT BMDM following engulfment of UV irradiated EL4-HBZ cells with no STAVs (62), WT BMDM following engulfment of UV irradiated EL4-HBZ cells transfected with STAVs (3 ug/ml) for 3 hours (63), SKO BMDM (54), SKO BMDM following engulfment of UV irradiated EL4-HBZ cells with no STAVs (64), and SKO BMDM following engulfment of UV irradiated EL4-HBZ cells transfected with STAVs (3 ug/ml) (64);

FIG. 5C shows a schematic representation of the dead cell immunization protocol, where EL4-HBZ cells were transfected with STAVs for 3 hours and UV irradiated (120 mJ/cm) (after 24 hours, wild type mice were injected i.p. with the irradiated EL4-HBZ with/without STAVs, after the primary injection, mice were boosted with the irradiated EL4-HBZ cells with/without STAVs at Day 21 and Day 42, at 3 weeks after the second boost, mice were challenged with EL4-HBZ cells (5×10 cells/mouse) on the flank);

FIG. 5D shows a Western blot analysis of HBZ (using anti-HBZ) and STING (using anti-STING) in EL4 cells (66) and EL4-HBZ cells (67);

FIG. 5E shows the tumor volumes for the mice treated as per FIG. 5C measured on the indicated days; PBS (16), irradiated EL4-HBZ (62), and irradiated EL4-HBZ with STAVs (63);

FIG. 5F shows the tumor volumes for the mice treated as per FIG. 5C measured on day 14 after the EL4-HBZ challenge; PBS (16), irradiated EL4-HBZ (62), and irradiated EL4-HBZ with STAVs (63);

FIG. 6A shows a schematic representation of the dead cell therapy in the murine AML model (C1498), where on Day 0, C1498 cells were inoculated s.c. in wild type C57/BL6 mice. C1498 cells were transfected with STAVs (3 μg/ml) for 3 hours and UV irradiated (120 mJ/cm), and incubated for 24 hours (mice were injected i.p. with the irradiated C1498 cells without STAVs (72), or irradiated C1498 cells transfected with STAVs three times, on Days 2, 5, and 10 (73), each treatment included different STAVs: STAV1 (A:T) on Day 2, STAV2 (AC:TG) on Day 5, and STAV3 (AT) on Day 10. PBS was used as a control (17)).

FIG. 6B shows the tumor volumes for the mice treated as per FIG. 6A measured on the indicated days; PBS (17), irradiated C1498 cells without STAVs (72), and irradiated C1498 cells without STAVs (73);

FIG. 6C shows the tumor weight for the mice treated as per FIG. 6A measured on day 16 after the C1498 challenge; PBS (17), irradiated C1498 cells without STAVs (72), and irradiated C1498 cells without STAVs (73);

FIG. 6D shows an indirect ELISA assay used for detection of STAV-specific antibodies from mouse sera (ELISA plates were pre-coated with STAVs (A:T) at 0.1 μg/ml. Sera from PBS treated (17), UV irradiated C1498 cells (75), and UV irradiated C1498 containing STAVs (76) were added to the wells. Anti-dsDNA (Abcam: ab27156) (74) was used as calibrator (standard curve), sera from untreated mice was used as a negative control (18);

FIG. 6E is a histogram showing FACS analysis for splenocytes isolated on Day 16 and stained with anti-CD45-PacificBlue and anti-CD19-AlexaFluor 700) to generate CD19 immune cell population as a percentage of CD45; PBS treated (17), UV irradiated C1498 cells (75), UV irradiated C1498 containing STAVs (76), and negative control (18);

FIG. 6F is a histogram showing FACS analysis for splenocytes isolated on Day 16 and stained with anti-CD45-PacificBlue and anti-CD3-FITC) to generate CD3 immune cell population as a percentage of CD45; PBS treated (17), UV irradiated C1498 cells (75), UV irradiated C1498 containing STAVs (76), and negative control (18);

FIG. 6G is a histogram showing FACS analysis for splenocytes isolated on Day 16 and stained with anti-CD3-FITC and anti-CD4-PE) to generate CD4 immune cell population as a percentage of CD3; PBS treated (17), UV irradiated C1498 cells (75), UV irradiated C1498 containing STAVs (76), and negative control (18);

FIG. 6H is a histogram showing FACS analysis for splenocytes isolated on Day 16 and stained with anti-CD8a-PercP and anti-CD3-FITC) to generate CD8 immune cell population as a percentage of CD3; PBS treated (17), UV irradiated C1498 cells (75), UV irradiated C1498 containing STAVs (76), and negative control (18);

FIG. 7A shows a schematic representation of the dead cell therapy in murine ALL model (EL4 cells). On Day 0, EL4 cells were inoculated s.c. in wild type C57/BL6 mice. STAVs were transfected using MaxCyte GT transfection system (MaxCyte, Gaithersburg, Md., USA). STAVs transfected-EL4 cells were UV irradiated (120 mJ/cm) and incubated for 24 hours. The mice were injected i.p. with UV irradiated EL4 cells with (74) and UV irradiated EL4 cells transfected with STAVs three times, on Days 2, 5, and 10 (75). Each treatment includes different STAVs: STAV1 (A:T) on Day 2, STAV2 (AC:TG) on Day 5, and STAV3 (AT) on Day 10, PBS was used as a control (18);

FIG. 7B shows the tumor volumes for the mice treated as per FIG. 7A measured on the indicated days;

FIG. 7C shows the tumor weight for the mice treated as per FIG. 7A measured on day 14 after the challenge;

FIG. 8A shows a Western blot analysis of p-TBK1, p-p65, p-IRF3, p-STING, cGAS and STING in human AML and ATLL (JAE) cells transfected with STAVs (3 μg/ml) at 6 hours; AML without STAVs (19), AML transfected with STAVs (84), ATLL without STAVs (19), and ATLL transfected with STAVs (85);

FIG. 8B shows a schematic representation of the phagocytosis of AML and ATLL cells by human macrophages derived from CD14+ monocytes (Lonza). AML and ATL (JAE) cells were transfected by STAVs for 3 hours and UV irradiated (120 mJ/cm). The irradiated AML and ATL (JAE) cells were fed to human macrophages at 24 hours after UV irradiation;

FIG. 8C is a histogram showing qPCR analysis of IFNβ in WT macrophages following engulfment of AML cells without STAVs (82) and with STAVs (84), where Mock is shown (19);

FIG. 8D is a histogram showing qPCR analysis of IFNβ in WT macrophages following engulfment of ATL (JAE) cells without STAVs (83) and with STAVs (85), where Mock is shown (20);

FIG. 9A is a flow diagram showing a treatment protocol for a cancer requiring treatment with a plurality of doses of leukemic cells treated with a plurality of STAVs selected from the group consisting of STAV 1, STAV 2, STAV 3, STAV 4 and STAV5, according to an embodiment of the invention;

FIG. 9B is a flow diagram showing a treatment protocol for a cancer requiring treatment with a plurality of doses of leukemic cells treated with a plurality of STAVs selected from the group consisting of STAV 1, STAV 2, STAV 3, STAV 4 and STAV5 and a treatment with a Dendritic Cell vaccine generated with at least one STAV, according to an embodiment of the invention;

FIG. 9C is a flow diagram showing a treatment protocol for a cancer requiring treatment with a plurality of doses of leukemic cells treated with a plurality of STAVs selected from the group consisting of STAV 1, STAV 2, STAV 3, STAV 4 and STAV5 and a treatment with a plurality of Dendritic Cell vaccines generated with a plurality of STAVs selected from the group consisting of STAV 1, STAV 2, STAV 3, STAV 4 and STAV5, according to an embodiment of the invention;

FIG. 9D is a flow diagram showing a treatment protocol for a cancer requiring treatment with a plurality of doses of leukemic cells treated with up to five STAVs comprising the group consisting of STAV 1, STAV 2, STAV 3, STAV 4 and STAV5 and a treatment with a plurality of Dendritic Cell vaccines generated with a plurality of STAVs selected from the group consisting of STAV 1, STAV 2, STAV 3, STAV 4 and STAV5, according to an embodiment of the invention; and

FIG. 10 is a flow diagram showing a limiting toxicity protocol for relapsed/refractory aggressive leukemia.

### DETAILED DESCRIPTION OF THE INVENTION

Described herein are methods and compositions for modulating an immune response in a subject having a disease or disorder associated with aberrant STING function. The below described preferred embodiments illustrate adaptation of these compositions and methods. Nonetheless, from the description of these embodiments, other aspects of the invention can be made and/or practiced based on the description provided below.

Methods and compositions for modulating an immune response in a subject (e.g., a human being, dog, cat, horse, cow, goat, pig, etc.) having a disease or disorder associated with aberrant STING function involve a pharmaceutical composition including an agent which modulates STING function and a pharmaceutically acceptable carrier, wherein amount the pharmaceutical composition is effective to ameliorate the aberrant STING function in the subject.

Diseases or disorders associated with aberrant STING function can be anywhere cells having defective STING function or expression cause or exacerbate the physical symptoms of the disease or disorder. Commonly, such diseases or disorders are mediated by immune system cells, e.g., an inflammatory condition, an autoimmune condition, cancer (e.g., breast, colorectal, prostate, ovarian, leukemia, lung, endometrial, or liver cancer), atherosclerosis, arthritis (e.g., osteoarthritis or rheumatoid arthritis), an inflammatory bowel disease (e.g., ulcerative colitis or Crohn's disease), a peripheral vascular disease, a cerebral vascular accident (stroke), one where chronic inflammation is present, one characterized by lesions having inflammatory cell infiltration, one where amyloid plaques are present in the brain (e.g., Alzheimer's disease), Aicardi-Goutieres syndrome, juvenile arthritis, osteoporosis, amyotrophic lateral sclerosis, or multiple sclerosis.

The agent can be a nucleic acid molecule that binds to STING under intracellular conditions (i.e., under conditions inside a cell where STING is normally located) having a molecular weight less than 20,000, daltons or less than 30,000 daltons that increases STING function or expression. The agent can also be a STING-binding nucleic acid molecule which can be a single-stranded (ss) or double-stranded (ds) RNA or DNA. Preferably the nucleic acid is 70 base pairs, or between 40 and 150, 60 and 120, 80 and 100, or 85 and 95 base pairs in length. The STING-binding nucleic acid molecule can be nuclease-resistant, e.g., made up of nuclease-resistant nucleotides or in cyclic dinucleotide form. The agent can also be associated with a molecule that facilitates transmembrane transport.

Methods and compositions for treating cancer in a subject having a cancerous tumor infiltrated with inflammatory immune cells involve a pharmaceutical composition including an agent which downregulates STING function or expression and a pharmaceutically acceptable carrier, wherein amount the pharmaceutical composition is effective to reduce the number of inflammatory immune cells infiltrating the cancerous tumor by at least 50% (e.g., at least 50, 60, 70, 80, or 90%, or until reduction of inflammatory cell infiltration is detectably reduced by histology or scanning).

The compositions described herein might be included along with one or more pharmaceutically acceptable carriers or excipients to make pharmaceutical compositions which can be administered by a variety of routes including oral, rectal, vaginal, topical, transdermal, subcutaneous, intravenous, intramuscular, insufflation, intrathecal, and intranasal administration. Suitable formulations for use in the present invention are found in Remington's Pharmaceutical Sciences, Mack Publishing Company, Philadelphia, Pa., 17th ed. (1985).

The active ingredient(s) can be mixed with an excipient, diluted by an excipient, and/or enclosed within a carrier which can be in the form of a capsule, sachet, paper or other container. When the excipient serves as a diluent, it can be a solid, semi-solid, or liquid material, which acts as a vehicle, carrier or medium for the active ingredient. The compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, sterile liquids for intranasal administration (e.g., a spraying device), or sterile packaged powders. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

For preparing solid formulations such as tablets, the composition can be mixed with a pharmaceutical excipient to form a solid preformulation composition containing a homogeneous mixture of a compound. Tablets or pills may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid forms of the formulations include suspensions and emulsions. To enhance serum half-life, the formulations may be encapsulated, introduced into the lumen of liposomes, prepared as a colloid, or incorporated in the layers of liposomes. A variety of methods are available for preparing liposomes, as described in, e.g., Szoka, et al., U.S. Pat. Nos. 4,235,871, 4,501,728 and 4,837,028 each of which is incorporated herein by reference in its entirety and for all purposes.

The compositions are preferably formulated in a unit dosage form of the active ingredient(s). The amount administered to the patient will vary depending upon what is being administered, the purpose of the administration, such as prophylaxis or therapy, the state of the patient, the manner of administration, and the like all of which are within the skill of qualified physicians and pharmacists. In therapeutic applications, compositions are administered to a patient already suffering from a disease in an amount sufficient to cure or at least partially arrest the symptoms of the disease and its complications. Amounts effective for this use will depend on the disease condition being treated as well as by the judgment of the attending clinician depending upon factors such as the severity of the symptoms, the age, weight and general condition of the patient, and the like.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

Regulation of the innate immune system to facilitate robust anti-tumor Cytotoxic T cell (CTL) responses is proving to be a powerful approach for the effective treatment of a variety of cancers. Key cellular innate immune sensors, such as STING (stimulator of interferon genes) have evolved to detect microbial infection through recognition of pathogen-derived nucleic acids, an event which triggers the transcription of numerous host defense-related proteins and pro-inflammatory cytokines. STING resides in the endoplasmic reticulum and is activated by cyclic dinucleotides (CDNs) such as cyclic di-GMP and cyclic-di-AMP secreted by intracellular bacteria following infection. Alternatively, STING can be activated by cyclic GMP-AMP (cGAMP) generated by a cellular cGAMP synthase (cGAS) after association with aberrant cytosolic dsDNA species, which can include microbial DNA or self-DNA leaked from the nucleus. Cytosolic dsDNA species present within a dying tumor cell can activate extrinsic STING signaling in phagocytes following association with cGAS which would generate CDNs. Generally, the cytosol of the cell is free of DNA, since it would aggravate STING-dependent cytokine production, an event that can lead to lethal auto inflammatory disease. For example, self-DNA leaked from the nucleus, following cell division or following DNA damage, is prevented from activating STING signaling by the exonuclease DNase III (Trex1). Consequently, defects in Trex1 function lead to severe auto inflammatory disease due to undigested self-DNA triggering STING activity. In addition, following the engulfment of apoptotic cells, phagocyte-dependent DNase II plays a critical role in digesting the DNA within the dead cell, to prevent it from activating STING-signaling extrinsically. Loss of DNase II function is embryonic lethal in murine models due to high-level cytokine production being instigated by overactive STING activity.

Tumor cells are notoriously non-immunogenic through their ability to mimic the properties of normal cells which have naturally evolved to avoid activating the immune system following cell death and phagocytosis. Apoptotic cells thus avoid activating extrinsic STING signaling following phagocytosis, to avoid harmful auto-inflammatory responses. In an embodiment of the present invention, a way of making ‘cold’ tumors ‘hot’ (highly immunogenic) was achieved by loading tumor cells with STAVs. STAVs are synthetic oligo dsDNA species of >70 nucleotides in length, which upon cell transfection predominantly reside in the cytosol and avoid nuclear DNases, responsible for degrading genomic DNA. Notably, STING signaling is critically important for facilitating anti-tumor T cell activity. In this scenario, tumor cells carrying STAVs potently activate STING in APCs, following engulfment. STAVs loaded syngeneic tumor cells inoculated in immunocompetent mice were able to generate anti-tumor T cell activity in vivo, and to prevent lethal metastatic disease. The stimulation of innate immune signaling pathways leading to cytokine production within phagocytes such as CD8+ dendritic cells (DCs) also involve STING. In an embodiment of the present invention, the sequential use of autologous leukemia cells loaded with different STAVs ex vivo, administered alone or concomitantly with a personalized dendritic cell (DC) vaccine (prepared from autologous DCs stimulated by STAVs loaded leukemic cells) can be used to activate STING.

In an embodiment of the present invention, patients can be inoculated with incurable treatment-refractory HTLV-1 associated ATLL, AML, and ALL with autologous STAV loaded, irradiated tumor cells, with the objective of generating anti-tumor immune response. This objective can include co-culturing autologous DC'S with STAV loaded tumor cells and inoculating the patient with already primed APC's.

DCs are specialized APCs found in blood and throughout most organ tissues. DCs strongly express major histocompatibility complex (MHC), adhesion, and co-stimulatory molecules necessary for the stimulation of T cell responses and adaptive cell immunity. DCs are located at sites of antigen capture and after they phagocyte pathogens, foreign antigens, or damaged cells they subsequently migrate to lymphatic areas for antigen presentation. By expressing both MHC class I and class II molecules, they can prime both cytotoxic CD8+ cells and CD4+ helper T-cells respectively, and both of these cell types are thought to be necessary for an effective cell-mediated immune response. DCs can also strongly activate NK and NK-T cells thus linking innate and adaptive immune responses, thus potentially targeting tumor cells for killing with and without expression of MHC class I molecules. DCs interact with foreign antigens ex vivo, and present these to naive CD4+ T cells to generate clonal expansion of effector T cells.

Cells are harvested from the patient and the cancer cells are separated, frozen into separate vials. One vial is thawed and transfected with one STAV. The transfected cells are irradiated with ultraviolet (UV) radiation, incubated, and re-infused into the patient. Three (3) to four (4) weeks later another vial is thawed and transfected with a different STAV. This process can be repeated another three (3) times. The method aims to prime and boost the patient to their own tumor. The more boosts, the stronger the immune response to the tumor. None of STAV1, STAV2, STAV3, STAV4 or STAV5 shares significant homology to human DNA. The STAVs are chosen in this way to minimize the risk of immunizing patients against their own DNA sequences. Each boost uses a different, unique STAV to avoid generating an immune response to the DNA/STAVs (an anti nuclear antibody (ANA)). If the same STAV was used five (5) times, a significant ANA response to the STAV can be generated. The method is thus geared to establishing an immune response only to the tumor cell (tumor antigen).

In an embodiment of the present invention, UV irradiation can be used to kill the cells. In an alternative embodiment of the present invention, X-rays or gamma emitters can be used to kill cells. In other embodiments of the present invention, any radiation source that would kill the cell can be used to treat the transfected cells.

STAV 5 is a sequence derived from intron sequences from the HSV1 genome (i.e., HSV RL2 intron-S and HSV RL2 intron-AS). It was chosen because the sequence has no similarity to human DNA as determined from computational analysis.

DC vaccines have emerged as promising cancer immunotherapy approaches. DC vaccines can be generated from large numbers of progenitor cells cultured ex vivo in the presence of cytokines after exposing these to foreign antigens. Tumor cells can evade immune recognition by blunting T cell responses via several mechanisms; these may include: 1) presenting tumor antigens in the relative absence of co-stimulatory molecules required for the activation of effector T cells thus inducing T cell anergy rather than immunity, 2) creating a micro-environment rich in immunosuppressive T-regulatory cells (Tregs) and myeloid derived suppressor cells, and 3) upregulating negative co-stimulatory pathways such as those mediated by CTLA-4 and PDL-1/PD-1 thus favoring tumor growth and survival. Malignant cells can also inhibit the function of DCs thus making them more tolerant to tumor antigens. Therefore, effective cancer vaccines require efficient presentation of tumor antigens, adequate co-stimulation leading to T-cell priming, and successful reversal of the immunosuppression induced by tumor cells in order to achieve long-term immunity. Animal models have demonstrated that DC tumor vaccines can reverse T-cell anergy resulting in subsequent tumor rejection. Several clinical trials and pre-clinical studies have evaluated DC vaccines against various cancers, including hematologic malignancies, and demonstrated safety. In one study, a personalized whole tumor cell fusion vaccine of Acute Myelogenous Leukemia (AML) and DCs elicited the expansion of leukemia-specific T cells and protected against disease relapse in elderly patients with AML. A recently tested DC vaccine consisting of autologous DCs pulsed with HTLV-1 Tax peptides corresponding to CTL epitopes was administered to three (3) pre-treated patients with HTLV-1 associated adult T-cell leukemia-lymphoma (ATLL), and two (2) patients survived for more than four (4) years after vaccination without severe adverse effects. DCs loaded with leukemia-derived apoptotic bodies from adult patients with ALL increased their ability to stimulate both allogeneic and autologous T lymphocytes, and to generate specific anti-leukemic CD3+ cells. These findings offered a rationale for designing DC-based vaccine approaches for patients with ALL with the objective of controlling/eradicating the disease. In an embodiment of the present invention, personalized serial injections of autologous mature DCs stimulated exogenously with patient's own leukemic cells loaded with STAVs for patients with incurable treatment-refractory HTLV-1 associated ATLL, AML, and ALL can be used.

The stimulation of innate immune signaling pathways leading to cytokine production within phagocytes such as CD8+ DCs involve STING. Recent clinical trials have demonstrated that adjuvant DC vaccines comprised of tumor antigen stimulated DCs are a safe, feasible, and potentially beneficial for some patients, however clinical responses using such approaches alone have only been modest. DNase-resistant nucleic acid-based STAVs are innate immune agonists that make previously immuno-evasive, inert tumor cells highly immunogenic via STING signaling in APCs, thus eliciting CTL priming followed by robust anti-tumor responses. Syngeneic tumor cells (including leukemias) loaded with STAVs render non-immunogenic cells ‘immunogenic’ and able to stimulate antigen presenting cells in vitro and in vivo. Immunocompetent mice bearing metastatic tumors can be cured following inoculation of syngeneic tumor cells loaded with STAVs. In an embodiment of the present invention, a cell-based immunotherapy approach of syngeneic tumor cells loaded with STAVs in combination with autologous DCs stimulated with STAVs loaded tumor cells for the treatment of deadly and incurable lymphoid and myelogenous leukemias. The use of STAVs alone, or in combination with other immunotherapy approaches can provide a powerful tool for use in treating cancer.

### EXAMPLES

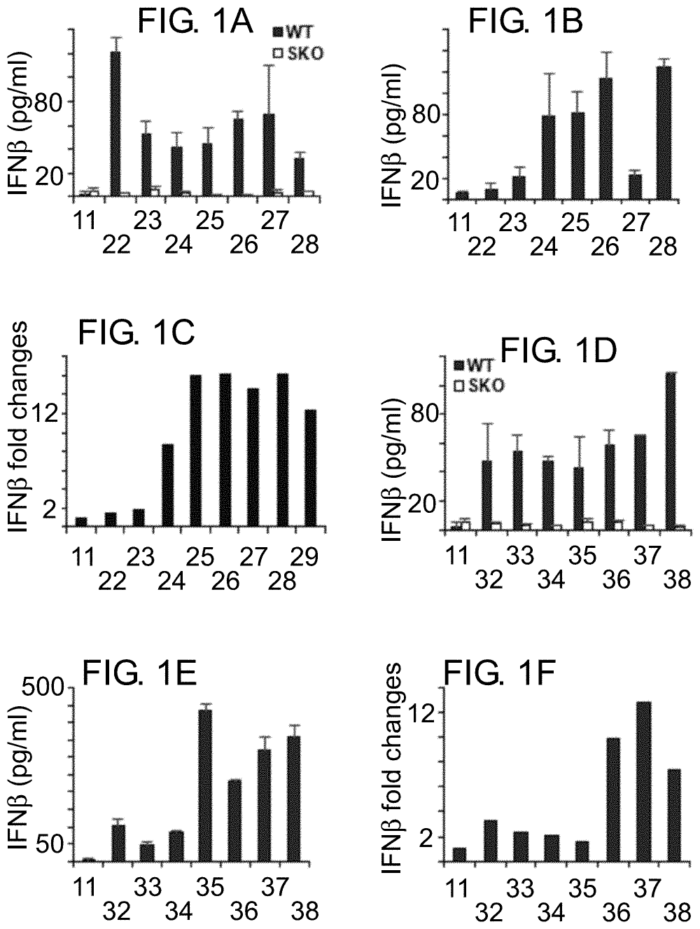

In an embodiment of the present invention, a variety of dsDNA and ssDNA species, that varied in their GC or AT content and evaluated which STAVs was better at stimulating STING signaling following transfection of normal human and mouse cells including APCs. In an embodiment of the present invention, the STAVs were synthetically generated and contained exonuclease resistant phosphorothioates at the ends (ES). STAVs that were greater than 70 bp were effective in stimulating STING-based cytokine production, regardless of nucleotide content (FIG. 1). FIGS. 1A-F show results for various STING ligands with different sequences and length. FIG. 1A and FIG. 1B show IFNβ ELISA assay in mouse embryonic fibroblasts (MEFs), hTERT transfected with different lengths of AT rich-STING ligands (A:T30ES (22), A:T50ES (23), A:T60ES (24), A:T70ES (25), A:T80ES (26), A:T90ES (27), and A:T100ES (28)). FIG. 1C shows qRT-PCR analysis of IFNβ1 in human macrophages transfected with different length of AT rich-STING ligands. FIG. 1D and FIG. 1E show IFNβ ELISA assays in MEFs, hTERT transfected with different lengths of GC rich-STING ligands (GC30ES (32), GC50ES (33), GC60ES (34), GC70ES (35), GC80ES (36), GC90ES (37), and GC100ES (38)). FIG. 1F shows qRT-PCR analysis of IFNβ1 in human macrophages transfected with different length of GC rich-STING ligands (GC30ES (32), GC50ES (33), GC60ES (34), GC70ES (35), GC80ES (36), GC90ES (37), and GC100ES (38)).

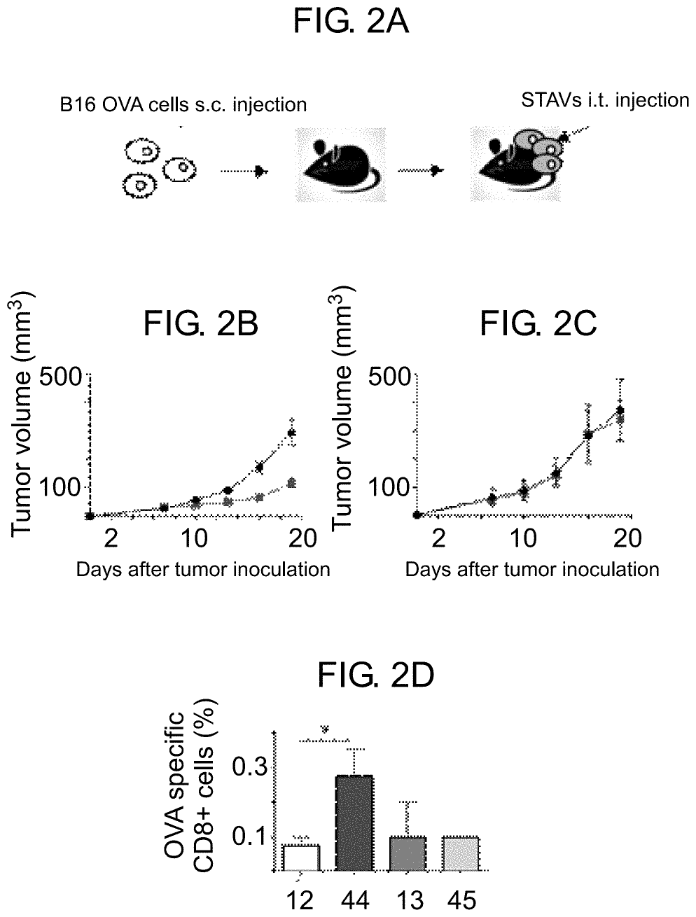

In an embodiment of the present invention, a first STAVs for primary inoculation (AT rich) can be used and a second STAVs for boosting purposes (GC) rich can be used to avoid autoimmune targeting of the STAV itself. In an embodiment of the present invention, AT rich STAVs (80 bp) are used. The STAVs were inoculated into tumors (B16-OVA) grown on the flanks of C57/BL6 mice. FIGS. 2A-D show significant anti-tumor activity of STAVs in B16 OVA melanomas bearing mice resulting in regression of tumors. FIG. 2A shows a schematic representation of intratumoral (i.t.) injection of STAVs in B16 OVA melanoma bearing mice. The mice were subcutaneously injected with B16-OVA cells on the flank. 10 ug of STAVs were injected i.t. every 3 days. The treatment show tumor volumes from WT (n=7/group) (see FIG. 2B) and STING knock out (SKO) mice (n=7/group) (see FIG. 2C) measured on the indicated days. FIG. 2D shows the frequency of OVA specific CD8+ T cells in the spleen from WT (n=4/group) and SKO (n=4/group) mice injected with STAV (44, 45) or PBS as control (12, 13) respectively.

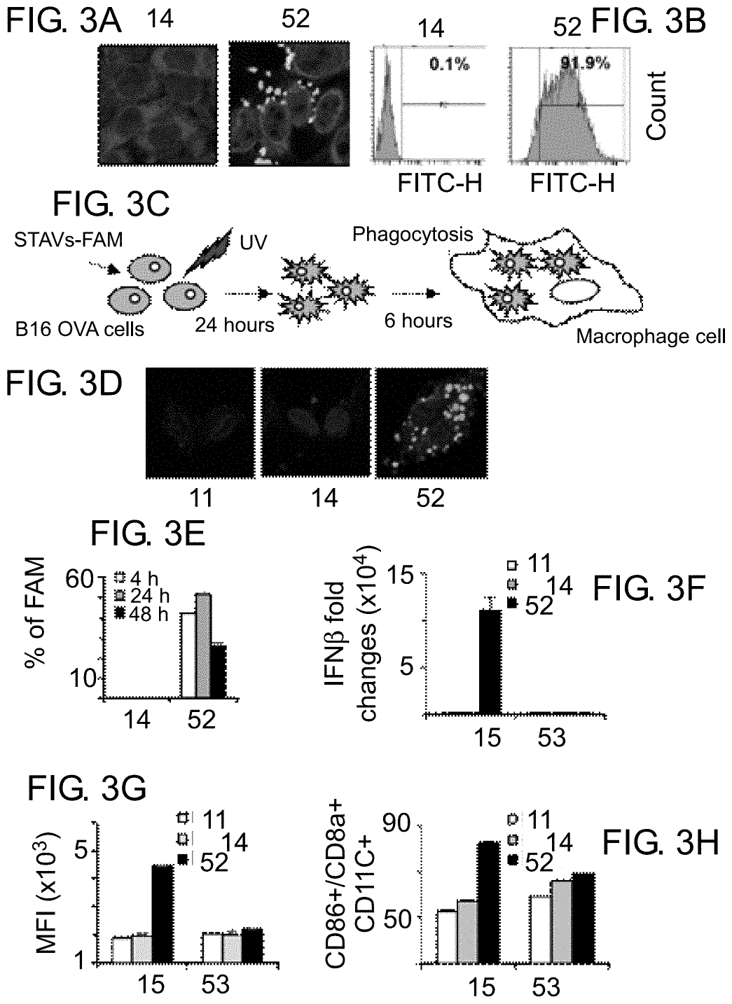

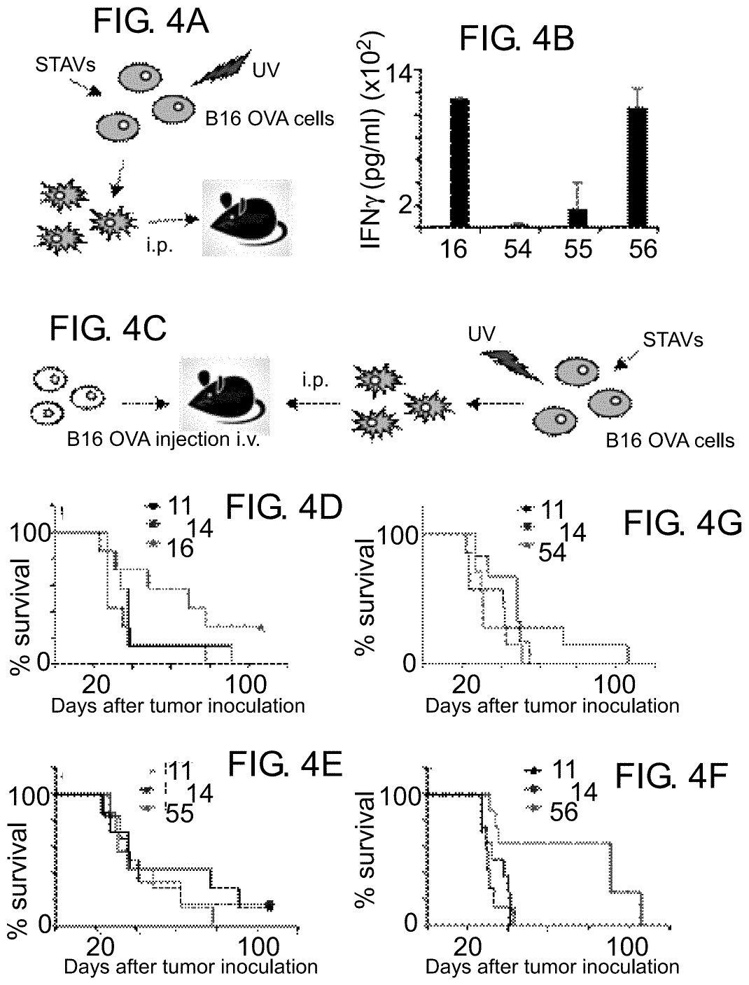

To complement this approach, tumor cells (B16 melanoma) were loaded with fluorescently labelled STAVs (polyA90ES-FAM and polyT90ES-FAM referred to as STAVs-FAM in FIG. 3 below). The STAV-FAM was labeled with fluorescein (FAM) at the 5′end (see SEQ ID NO:35, SEQ ID NO:36). The STAV-FAMs were used to visualize STAV. Greater than 90% of the B16 cells took up the STAVs following transfection (FIG. 3A). FIG. 3A shows confocal analyses of B16 OVA cells (B16) transfected with FAM labeled STAVs (staining appears as small white moieties present in 52 but not 14, around dark cell bodies (stained with anti-DAPI) (in both 14 and 52) whose outer surface (stained with anti-calreticulin) (in both 14 and 52) appears as a grey halo around the dark cells; bar size, 10 μm (see Ahn et al., Cancer Cell, 33 (2018) 862-873 (FIG. 1A) which is incorporated by reference in its entirety and for all purposes). FIG. 3B shows fluorescence-activated cell sorting (FACS) analyses of B16 OVA cells (B16) transfected with FAM labeled STAVs. Further, the nuclease resistant STAVs within the B16 cells were efficiently engulfed by macrophages and were retained for up to 48 hours (FIG. 3C-E). The irradiated B16 cells were fed to macrophages at 24 hr after UV irradiation. Confocal Microscopy analysis (FIG. 3D) and FACS (FIG. 3E) in macrophages following cellular engulfment of B16 cells transfected with FAM labeled STAVs. FIG. 3F shows qRT-PCR analysis of IFNβ1 in wild type (WT) and STING knock out (SKO) macrophages (WT macrophages and SKO macrophages) following engulfment of B16 cells in presence or absence of STAVs. FIG. 3G shows FACS analysis for CD86 on macrophages (CD11b+) following phagocytosis of B16 cells. FIG. 3H shows FACS analysis for CD86 on CD8a+CD11C+ dendritic cells following phagocytosis of B16 cells containing STAVs. This enables the STAVs to be released within the macrophages and activate cGAS, resulting in the generation of CDNs and STING signaling. This is demonstrated by observing that macrophages phagocytosing B16 cells only containing STAVs were able to stimulate the production of type I IFN (see FIG. 3F). This was completely dependent on extrinsic STING signaling within the macrophages (SKO=STING-deficient macrophages). This strategy also caused the upregulation of maturation markers on macrophages or dendritic cells (DCs) in a STAVs-dependent, STING-dependent manner (see FIG. 3G and FIG. H). This was also seen in human APCs subsets. While AT rich STAVs in B16 cells were seen to stimulate APC's in trans, the effect was not observed using B16 cells loaded with dsRNA or CDNs. This is likely due to the cells being degraded prior to exerting activity. The next key question addressed was whether the STAVs loaded-UV treated B16 cells were immunogenic and exerted potent anti-tumor activity in vivo. To evaluate C57/BL6 mice were inoculated with syngeneic B16 tumors, through the tail vein. Such B16 colonize to the lungs and cause fatal metastatic disease within 30 days or less. Once established, such tumors are extremely hard to eliminate via therapeutic intervention. However, B16 cells loaded with STAVs can significantly prolong the survival of C57 mice carrying metastatic B16 cancer (the B16 cells also expressed ovalbumin so T cell activity to OVA antigen can be measured). B16-STAV treated mice generated high IFN-γ levels to OVA. Using mice deficient in STING signaling, or TLR9, CTL activity was dependent on STING signaling in APC's, but independent of TLR9. FIG. 4A shows a schematic representation of the dead cell immunization protocol where B16 OVA cells were transfected with STAVs for 3 hours, then UV irradiated (120 mJ/cm), incubated for 24 hours and then the cells were i.p. injected into mice. FIG. 4B shows an IFNγ measurement in splenocytes where B16 OVA cells were transfected with STAVs for 3 hours, then UV irradiated (120 mJ/cm), incubated for 24 hours and then the dead cells were i.p. injected into WT (16), SKO (54), cGAS KO (55), and TLR9 KO (56) mice at 7 days after the second immunization, error bars indicate mean±SD.

FIG. 4C shows a schematic representation of post-vaccination protocol for B16 OVA mediated lung metastasis in mice. FIG. 4D is a histogram showing post-vaccination survival rates for B16 OVA mediated lung metastasis from WT mice (16) i.v. injected with B16 OVA cells (5×104 cells/mouse), on days 1, 3, 7, and 14, mice were i.p. injected with UV irradiated B16 OVA cells (1×106 cells/mouse) with STAVs (p=0.0429, n=7/group, p values were based on Log-rank test, with p<0.05 considered statistically significant), where PBS treated (11) and B16 with no DNA transfected treated (14) are also shown. FIG. 4E is a histogram showing post-vaccination survival rates for B16 OVA mediated lung metastasis from cGAS KO mice (55) i.v. injected with B16 OVA cells (5×104 cells/mouse), on days 1, 3, 7, and 14, mice were i.p. injected with UV irradiated B16 OVA cells (1×10 cells/mouse) with STAVs (p=0.4075, n=7/group, p values were based on Log-rank test, with p<0.05 considered statistically significant), where PBS treated (11) and B16 with no DNA transfected treated (14) are also shown. FIG. 4F is a histogram showing post-vaccination survival rates for B16 OVA mediated lung metastasis from TLR9 KO mice (56) i.v. injected with B16 OVA cells (5×104 cells/mouse), on days 1, 3, 7, and 14, mice were i.p. injected with UV irradiated B16 OVA cells (1×106 cells/mouse) with STAVs (p=0.0012, n=7/group, p values were based on Log-rank test, with p<0.05 considered statistically significant), where PBS treated (11) and B16 with no DNA transfected treated (14) are also shown. Accordingly, mice lacking STING or cGAS did not exert B16 specific anti-tumor activity (see FIG. 4C-F).

FIG. 4G is a histogram showing post-vaccination survival rates for B16 OVA mediated lung metastasis from SKO mice (54) i.v. injected with B16 OVA cells (5×104 cells/mouse), on days 1, 3, 7, and 14, mice were i.p. injected with UV irradiated B16 OVA cells (1×106 cells/mouse) with STAVs (p=0.2616, n=7/group, p values were based on Log-rank test, with p<0.05 considered statistically significant), where PBS treated (11) and B16 with no DNA transfected treated (14) are also shown. However, the effects were independent of TLR9 (see FIG. 4G).

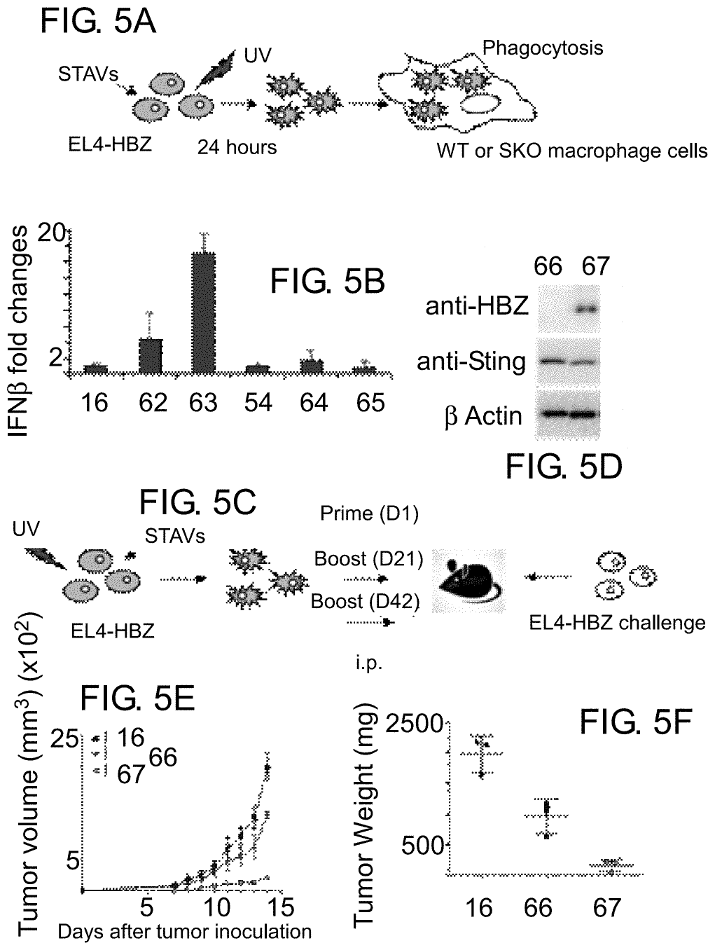

An anti-tumor therapy against re-infusible tumors, such as leukemias by treating patient's tumors with STAVs, irradiating, and re-infusing. The tumor cells can be engulfed by APC's and the tumor specific proteins presented on MHC can prime anti-tumor T cells. FIG. 5D shows a Western blot analysis of HBZ (using anti-HBZ) and STING (using anti-STING) in EL4 cells (66) and EL4-HBZ cells (67). Syngeneic tumors models for EL4 leukemia (ALL model) expressing HTLV-1 bZip factor (HBZ) (see FIG. 5D) in immunocompetent mice (C57/BL6) have been established. FIG. 5A shows a schematic representation of the phagocytosis of EL4-HBZ cells by Bone Marrow Derived Macrophages (BMDM) from WT or SKO mice; EL4-HBZ cells (1×10⁶) were transfected with STAVs (3 ug/ml) for 3 hours and irradiated by UV (120 mJ/cm) (at 24 hours after UV irradiation, the irradiated EL4-HBZ cells were fed to WT macrophages (WT BMDM) or SKO macrophages (SKO BMDM)). FIG. 5B shows qPCR analysis of IFNβ in WT BMDM (16), WT BMDM following engulfment of UV irradiated EL4-HBZ cells with no STAVs (62), WT BMDM following engulfment of UV irradiated EL4-HBZ cells transfected with STAVs (3 ug/ml) for 3 hours (63), SKO BMDM (54), SKO BMDM following engulfment of UV irradiated EL4-HBZ cells with no STAVs (64), and SKO BMDM following engulfment of UV irradiated EL4-HBZ cells transfected with STAVs (3 ug/ml) (64). EL4 grows in the flanks of C57/BL6 mice or can metastasize to the lungs. EL4-HBZ cells loaded with STAVs potently activate APCs in a STING-dependent manner (see FIGS. 5A and B).

FIG. 5C shows a schematic representation of the dead cell immunization protocol, where EL4-HBZ cells were transfected with STAVs for 3 hours and UV irradiated (120 mJ/cm) (after 24 hours, wild type mice were injected i.p. with the irradiated EL4-HBZ with/without STAVs, after the primary injection, mice were boosted with the irradiated EL4-HBZ cells with/without STAVs at Day 21 and Day 42, at 3 weeks after the second boost, mice were challenged with EL4-HBZ cells (5×10 cells/mouse) on the flank). Irradiated control or STAV loaded EL4-HBZ cells were subsequently used to immunize C57/BL6 mice (6-8 weeks old; sex matched) (see FIG. 5C). Immunized mice were then challenged with live EL4-HBZ cells (s.c).

FIG. 5E shows the tumor volumes for the mice treated as per FIG. 5C measured on the indicated days; PBS (16), irradiated EL4-HBZ (62), and irradiated EL4-HBZ with STAVs (63). FIG. 5F shows the tumor volumes for the mice treated as per FIG. 5C measured on day 14 after the EL4-HBZ challenge; PBS (16), irradiated EL4-HBZ (62), and irradiated EL4-HBZ with STAVs (63). Only mice immunized with STAV loaded EL4-HBZ cells were protected from tumor growth (see FIGS. 5E and F) showing protection against EL4-HBZ tumor growth. Such protection can also apply to HTLV-1 associated ATLL, for which there are no good representative laboratory models as it only grows rarely in immunocompromised mice.

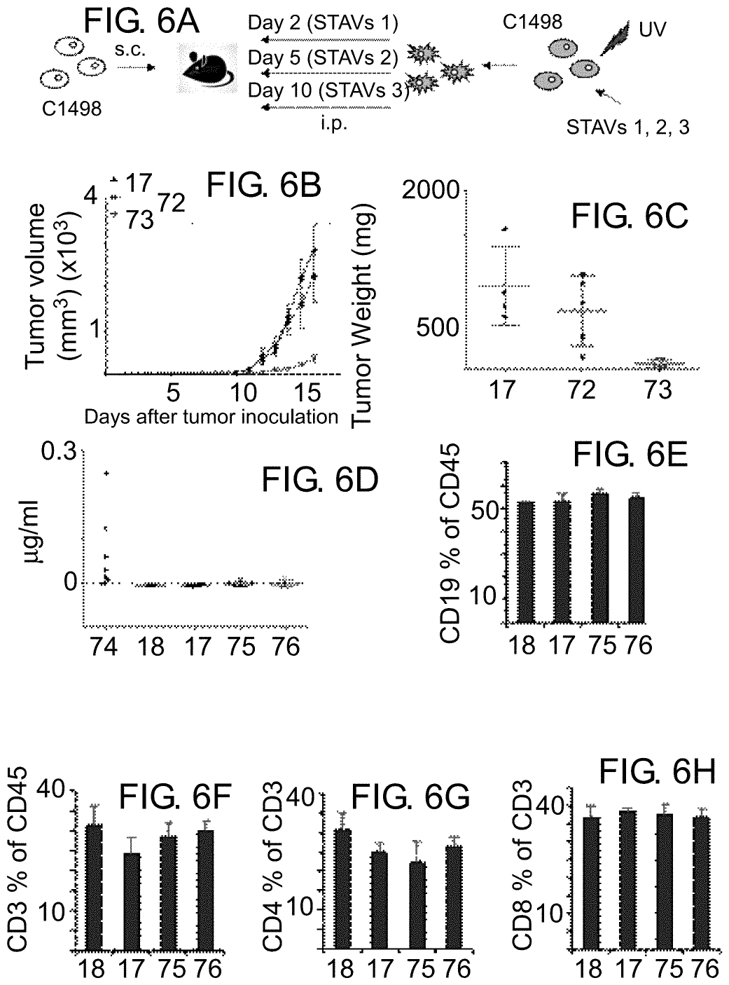

FIG. 6A shows a schematic representation of the dead cell therapy in the murine AML model (C1498), where on Day 0, C1498 cells were inoculated s.c. in wild type C57/BL6 mice. C1498 cells were transfected with STAVs (3 μg/ml) for 3 hours and UV irradiated (120 mJ/cm), and incubated for 24 hours (mice were injected i.p. with the irradiated C1498 cells without STAVs (72), or irradiated C1498 cells transfected with STAVs three times, on Days 2, 5, and 10 (73), each treatment included different STAVs: STAV1 (A:T) on Day 2, STAV2 (AC:TG) on Day 5, and STAV3 (AT) on Day 10. PBS was used as a control (17)). FIG. 6B shows the tumor volumes for the mice treated as per FIG. 6A measured on the indicated days; PBS (17), irradiated C1498 cells without STAVs (72), and irradiated C1498 cells without STAVs (73). FIG. 6C shows the tumor weight for the mice treated as per FIG. 6A measured on day 16 after the C1498 challenge; PBS (17), irradiated C1498 cells without STAVs (72), and irradiated C1498 cells without STAVs (73). Studies targeting AML. C1498 murine AML cell (expressing OVA; 5×10⁵) were inoculated into mice. After 2 days, STAV therapy was administered sequentially as shown in FIG. 6 (irradiated C1486-OVA plus STAV1 (A:T-76ES) followed by irradiated C1486 carrying STAV2 (AC:TG-76ES) on day 5, followed by irradiated C1486 carrying STAV3 (AT:TA-76ES) on day 10. Only cells carrying STAVs were able to therapeutically impair the growth of tumors (see FIG. 6A-C).

FIG. 6D shows an indirect ELISA assay used for detection of STAV-specific antibodies from mouse sera (ELISA plates were pre-coated with STAVs (A:T) at 0.1 μg/ml. Sera from PBS treated (17), UV irradiated C1498 cells (75), and UV irradiated C1498 containing STAVs (76) were added to the wells. Anti-dsDNA (Abeam: ab27156) (74) was used as calibrator (standard curve), sera from untreated mice was used as a negative control (18). At the end of the experiment (when tumors became large, the mice were sacrificed) their sera were analyzed for evidence of antibodies to dsDNA and more specifically to the administered STAVs. No evidence of anti-STAV antibodies in the EL4-STAV treated mice was found. (see FIG. 6D).

FIG. 6E is a histogram showing FACS analysis for splenocytes isolated on Day 16 and stained with anti-CD45-PacificBlue and anti-CD19-AlexaFluor 700) to generate CD19 immune cell population as a percentage of CD45; PBS treated (17), UV irradiated C1498 cells (75), UV irradiated C1498 containing STAVs (76), and negative control (18). Further, no evidence of aberrancies in the hematopoietic subsets from a variety of immune cells from the treated mice was found (see FIG. 6E).

FIG. 6F is a histogram showing FACS analysis for splenocytes isolated on Day 16 and stained with anti-CD45-PacificBlue and anti-CD3-FITC) to generate CD3 immune cell population as a percentage of CD45; PBS treated (17), UV irradiated C1498 cells (75), UV irradiated C1498 containing STAVs (76), and negative control (18). FIG. 6G is a histogram showing FACS analysis for splenocytes isolated on Day 16 and stained with anti-CD3-FITC and anti-CD4-PE) to generate CD4 immune cell population as a percentage of CD3; PBS treated (17), UV irradiated C1498 cells (75), UV irradiated C1498 containing STAVs (76), and negative control (18). FIG. 6H is a histogram showing FACS analysis for splenocytes isolated on Day 16 and stained with anti-CD8a-PercP and anti-CD3-FITC) to generate CD8 immune cell population as a percentage of CD3; PBS treated (17), UV irradiated C1498 cells (75), UV irradiated C1498 containing STAVs (76), and negative control (18).

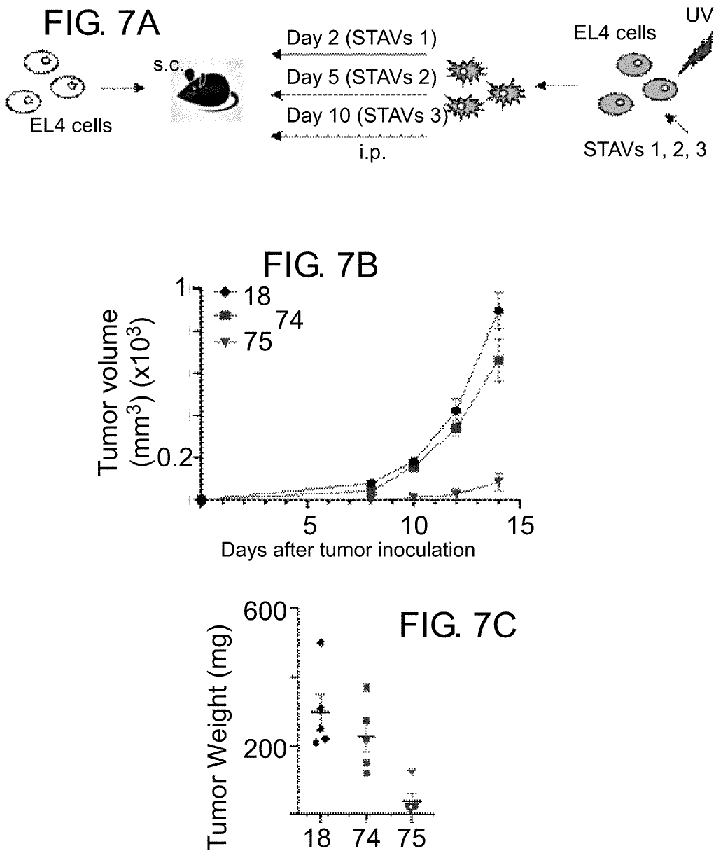

FIG. 7A shows a schematic representation of the dead cell therapy in murine ALL model (EL4 cells). On Day 0, EL4 cells were inoculated s.c. in wild type C57/BL6 mice. STAVs were transfected using MaxCyte GT transfection system (MaxCyte, Gaithersburg, Md., USA). STAVs transfected-EL4 cells were UV irradiated (120 mJ/cm) and incubated for 24 hours. The mice were injected i.p. with UV irradiated EL4 cells with (74) and UV irradiated EL4 cells transfected with STAVs three times, on Days 2, 5, and 10 (75). Each treatment includes different STAVs: STAV1 (A:T) on Day 2, STAV2 (AC:TG) on Day 5, and STAV3 (AT) on Day 10, PBS was used as a control (18). FIG. 7B shows the tumor volumes for the mice treated as per FIG. 7A measured on the indicated days. FIG. 7C shows the tumor weight for the mice treated as per FIG. 7A measured on day 14 after the challenge. Collectively, the data suggest no auto-immune responses to the therapy. These experiments were replicated using sequential administration of STAVs in the EL4 model and the same results were obtained demonstrating that only EL4 cells carrying STAVs impaired tumor growth (see FIG. 7).

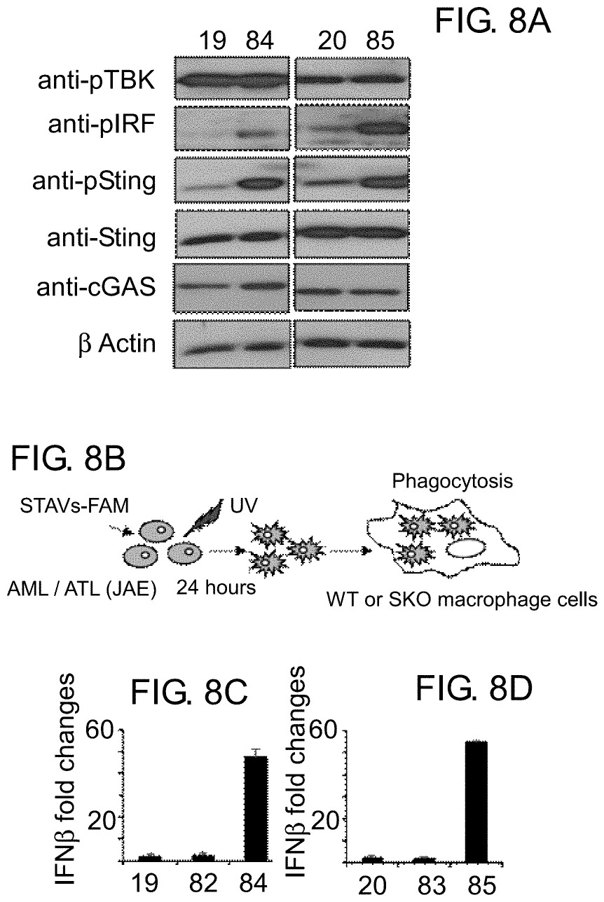

FIG. 8A shows a Western blot analysis of p-TBK1, p-p65, p-IRF3, p-STING, cGAS and STING in human AML and ATLL (JAE) cells transfected with STAVs (3 μg/ml) at 6 hours; AML without STAVs (19), AML transfected with STAVs (84), ATLL without STAVs (19), and ATLL transfected with STAVs (85). FIG. 8B shows a schematic representation of the phagocytosis of AML and ATLL cells by human macrophages derived from CD14+ monocytes (Lonza). AML and ATL (JAE) cells were transfected by STAVs for 3 hours and UV irradiated (120 mJ/cm). The irradiated AML and ATL (JAE) cells were fed to human macrophages at 24 hours after UV irradiation. FIG. 8C is a histogram showing qPCR analysis of IFNβ in WT macrophages following engulfment of AML cells without STAVs (82) and with STAVs (84), where Mock is shown (19). FIG. 8D is a histogram showing qPCR analysis of IFNβ in WT macrophages following engulfment of ATL (JAE) cells without STAVs (83) and with STAVs (85), where Mock is shown (20). To further prove the validity of the strategy human AML cells (AML-193; ATCC CRL 9589) and HTLV-1/ATLL cells transfected with STAVs (A:T-76ES) were demonstrated to became highly immunogenic and able to activate APCs (human macrophages), similar to the findings using murine models. Transfected STAVs activated STING signaling in the tumor cells and following irradiation potently stimulated cytokine production, in trans, following phagocytosis (see FIG. 8).

Day 1: Subjects can undergo leukapheresis in order to obtain 200-300 ml plasma fraction enriched with peripheral blood mononuclear cells (PBMCs) for purification of leukemic cells and monocytes (target yield 5-30×109 cells).

Transfection of autologous leukemic cells loaded with STAVs. Leukemic cells can be separately transfected (loaded) ex vivo with STAVs 1, 2, 3, and 5, followed by UV irradiation. Five distinct STAVs sequences are shown below [synthesized by Trilink Biotechnologies, HPLC Purification Endotoxin Tested (<5 EU/ml), (ps) (indicates phosphorothiote linkage).

DNA vaccine: Mice were immunized with a plasmid encoding OVA by intramuscular (i.m.) electroporation (100 μg per mouse). The booster immunization was given by i.m. two (2) to four (4) weeks after the primary immunization. STING deficient animals (−/−) or controls (+/+) have been twice immunized twice using intramuscularly [i.m.] electroporation with a DNA vaccine encoding ovalbumin. Serum was measured for anti-OVA IgG. To evaluate if STING played a role in this signaling pathway, STING −/− or control mice were immunized with plasmid DNA encoding the ovalbumin gene. While normal B and T cell subsets were noted in unstimulated STING −/− animals, following immunization Sting −/− mice exhibited significantly less serum ovalbumin (OVA) specific immunoglobulin (Ig)G's compared to controls. In addition, spleen CD8 T-cell frequency and IFN-γ secretion was markedly reduced in Sting mice following immunization, compared to wild type mice. Since immunoglobulin responses to OVA peptide were normal, these data emphasized that the STING-governed DNA sensor pathway is essential for efficient DNA vaccine-induced T-cell responses to antigen. Given this information, it was evaluated whether STING played a role in facilitating T-cell responses following infection with the DNA virus vaccinia that expresses ovalbumin (VV-OVA). This study indicated that control mice, but not Sting (−/−) mice elicited strong T-cell responses to viral encoded OVA, verifying the importance of STING in innate immune signaling processes required for DNA adjuvant activity.

STING also appears important for recognizing DNA's ability to stimulate the innate immune response, including DNA comprising vectors, plasmids, poly dA-dT, poly dC-dG and DNA of varying lengths and sequence composition including ISD. Thus, in another preferred embodiment, STING modulates the innate immune response. It is concluded that STING may play a more predominant role in facilitating RIG-1 mediated innate signaling rather than MDA5. Interestingly, a significant defect was not detected (>5-fold) in the ability of transfected B-form DNA, i.e., poly dA-dT or non CpG containing ISD to induce IFNβ in MEFs lacking STING compared to controls.

A: T30ES
polyA30ES
(SEQ ID NO: 1)
A(ps)A(ps)A(ps)AAAAAAAAAAAAAAAAAAAAAAAA(ps)A(ps)
A(ps)A
polyT30ES
(SEQ ID NO: 2)
T(ps)T(ps)T(ps)TTTTTTTTTTTTTTTTTTTTTTTT(ps)T(ps)
T(ps)T
A: T50ES
polyA50ES
(SEQ ID NO: 3)
A(ps)A(ps)A(ps)AAAAAAAAAAAAAAAAAAAAAAAAAAAAAAAAAAAA
AAAAAAAA(ps)A(ps)A(ps)A
polyT50ES
(SEQ ID NO: 4)
T(ps)T(ps)T(ps)TTTTTTTTTTTTTTTTTTTTTTTTTTTTTTTTTTTT
TTTTTTTT(ps)T(ps)T(ps)T
A: T60ES
polyA60ES
(SEQ ID NO: 5)
A(ps)A(ps)A(ps)AAAAAAAAAAAAAAAAAAAAAAAAAAAAAAAAAAAA
AAAAAAAAAAAAAAAAAA(ps)A(ps)A(ps)A
polyT60ES
(SEQ ID NO: 6)
T(ps)T(ps)T(ps)TTTTTTTTTTTTTTTTTTTTTTTTTTTTTTTTTTTT
TTTTTTTTTTTTTTTTTT(ps)T(ps)T(ps)T
A: T70ES
polyA70ES
(SEQ ID NO: 7)
A(ps)A(ps)A(ps)AAAAAAAAAAAAAAAAAAAAAAAAAAAAAAAAAAAA
AAAAAAAAAAAAAAAAAAAAAAAAAAAA(ps)A(ps)A(ps)A
polyT70ES
(SEQ ID NO: 8)
T(ps)T(ps)T(ps)TTTTTTTTTTTTTTTTTTTTTTTTTTTTTTTTTTTT
TTTTTTTTTTTTTTTTTTTTTTTTTTTT(ps)T(ps)T(ps)T
A: T80ES
polyA80ES
(SEQ ID NO: 9)
A(ps)A(ps)A(ps)AAAAAAAAAAAAAAAAAAAAAAAAAAAAAAAAAAAA
AAAAAAAAAAAAAAAAAAAAAAAAAAAAAAAAAAAAAA(ps)A(ps)
A(ps)A
polyT80ES
(SEQ ID NO: 10)
T(ps)T(ps)T(ps)TTTTTTTTTTTTTTTTTTTTTTTTTTTTTTTTTTTT
TTTTTTTTTTTTTTTTTTTTTTTTTTTTTTTTTTTTTT(ps)T(ps)
T(ps)T
A: T90ES
polyA90ES
(SEQ ID NO: 11)
A(ps)A(ps)A(ps)AAAAAAAAAAAAAAAAAAAAAAAAAAAAAAAAAAAA
AAAAAAAAAAAAAAAAAAAAAAAAAAAAAAAAAAAAAAAAAAAAAAA
A(ps)A(ps)A(ps)A
polyT90ES
(SEQ ID NO: 12)
T(ps)T(ps)T(ps)TTTTTTTTTTTTTTTTTTTTTTTTTTTTTTTTTTTT
TTTTTTTTTTTTTTTTTTTTTTTTTTTTTTTTTTTTTTTTTTTTTTT
T(ps)T(ps)T(ps)T
A: T100ES
polyA100ES
(SEQ ID NO: 13)
A(ps)A(ps)A(ps)AAAAAAAAAAAAAAAAAAAAAAAAAAAAAAAAAAAA
AAAAAAAAAAAAAAAAAAAAAAAAAAAAAAAAAAAAAAAAAAAAAAAAAAA
AAAAAAA(ps)A(ps)A(ps)A
polyT100ES
(SEQ ID NO: 14)
T(ps)T(ps)T(ps)TTTTTTTTTTTTTTTTTTTTTTTTTTTTTTTTTTTT
TTTTTTTTTTTTTTTTTTTTTTTTTTTTTTTTTTTTTTTTTTTTTTTTTTT
TTTTTTT(ps)T(ps)T(ps)T
A: T110ES
polyA110ES
(SEQ ID NO: 15)
A(ps)A(ps)A(ps)AAAAAAAAAAAAAAAAAAAAAAAAAAAAAAAAAAAA
AAAAAAAAAAAAAAAAAAAAAAAAAAAAAAAAAAAAAAAAAAAAAAAAAAA
AAAAAAAAAAAAAAAAA(ps)A(ps)A(ps)A
polyT110ES
(SEQ ID NO: 16)
T(ps)T(ps)T(ps)TTTTTTTTTTTTTTTTTTTTTTTTTTTTTTTTTTTT
TTTTTTTTTTTTTTTTTTTTTTTTTTTTTTTTTTTTTTTTTTTTTTTTTTT
TTTTTTTTTTTTTTTTT(ps)T(ps)T(ps)T
GC30ES
(SEQ ID NO: 17)
G(ps)C(ps)G(ps)CGCGCGCGCGCGCGCGCGCGCGCG(ps)C(ps)
G(ps)C
GC50ES
(SEQ ID NO: 18)
G(ps)C(ps)G(ps)CGCGCGCGCGCGCGCGCGCGCGCGCGCGCGCGCGCG
CGCGCGCG(ps)C(ps)G(ps)C
GC60ES
(SEQ ID NO: 19)
G(ps)C(ps)G(ps)CGCGCGCGCGCGCGCGCGCGCGCGCGCGCGCGCGCG
CGCGCGCGCGCGCGCGCG(ps)C(ps)G(ps)C
GC70ES
(SEQ ID NO: 20)
G(ps)C(ps)G(ps)CGCGCGCGCGCGCGCGCGCGCGCGCGCGCGCGCGCG
CGCGCGCGCGCGCGCGCGCGCGCGCGCG(ps)C(ps)G(ps)C
GC80ES
(SEQ ID NO: 21)
G(ps)C(ps)G(ps)CGCGCGCGCGCGCGCGCGCGCGCGCGCGCGCGCGCG
GCGCGCGCGCGCGCGCGCGCGCGCGCGCGCGCGCGCG(ps)C(ps)G(ps)
C
GC90ES
(SEQ ID NO: 22)
G(ps)C(ps)G(ps)CGCGCGCGCGCGCGCGCGCGCGCGCGCGCGCGCGCG
CGCGCGCGCGCGCGCGCGCGCGCGCGCGCGCGCGCGCGCGCGCGCGC
G(ps)C(ps)G(ps)C
GC100ES
(SEQ ID NO: 23)
G(ps)C(ps)G(ps)CGCGCGCGCGCGCGCGCGCGCGCGCGCGCGCGCGCG
CGCGCGCGCGCGCGCGCGCGCGCGCGCGCGCGCGCGCGCGCGCGCGCGCGC
GCGCGCG(ps)C(ps)G(ps)C
STAV 1 (Poly A: T76 ES)
Poly A76 ES
(SEQ ID NO: 24)
A(ps)A(ps)A(ps)AAAAAAAAAAAAAAAAAAAAAAAAAAAAAAAAAAAA
AAAAAAAAAAAAAAAAAAAAAAAAAAAAAAAAAA(ps)A(ps)A(ps)A
Poly T76 ES
(SEQ ID NO: 25)
T(ps)T(ps)T(ps)TTTTTTTTTTTTTTTTTTTTTTTTTTTTTTTTTTTT
TTTTTTTTTTTTTTTTTTTTTTTTTTTTTTTTTT(ps)T(ps)T(ps)T
STAV 2 (Poly AC: TG76 ES)
PolyAC76ES
(SEQ ID NO: 26)
A(ps)C(ps)A(ps)CACACACACACACACACACACACACACACACACACA
CACACACACACACACACACACACACACACACACA(ps)C(ps)A(ps)C
PolyTG76ES
(SEQ ID NO: 27)
G(ps)T(ps)G(ps)TGTGTGTGTGTGTGTGTGTGTGTGTGTGTGTGTGTG
TGTGTGTGTGTGTGTGTGTGTGTGTGTGTGTGTG(ps)T(ps)G(ps)T
STAV 3 (Poly AT76 ES)
Poly AT76 ES
(SEQ ID NO: 28)
A(ps)T(ps)A(ps)TATATATATATATATATATATATATATATATATATA
TATATATATATATATATATATATATATATATATA(ps)T(ps)A(ps)T
AT76 ES
(SEQ ID NO: 29)
T(ps)A(ps)T(ps)ATATATATATATATATATATATATATATATATATAT
ATATATATATATATATATATATATATATATATAT(ps)A(ps)T(ps)A
STAV 4 (ACTG76 ES)
Poly ACTG76 ES
(SEQ ID NO: 30)
A(ps)C(ps)T(ps)GACTGACTGACTGACTGACTGACTGACTGACTGACT
GACTGACTGACTGACTGACTGACTGACTGACTGA(ps)C(ps)T(ps)G
Poly CAGT76 ES
(SEQ ID NO: 31)
C(ps)A(ps)G(ps)TCAGTCAGTCAGTCAGTCAGTCAGTCAGTCAGTCAG
TCAGTCAGTCAGTCAGTCAGTCAGTCAGTCAGTC(ps)A(ps)G(ps)T
STAV 5 (HSV RL2 intron)
HSV RL2 intron-S
(SEQ ID NO: 32)
G(ps)A(ps)C(ps)CCTATCGATACAGGGCACGGGGTCGAACTGTTGGGT
TTCGCCATGGTACCCCCTGCATTTATATAGCCAG(ps)A(ps)C(ps)C
HSV RL2 intron-AS
(SEQ ID NO: 33)
G(ps)G(ps)T(ps)CTGGCTATATAAATGCAGGGGGTACCATGGCGAAAC
CCAACAGTTCGACCCCGTGCCCTGTATCGATAGG(ps)G(ps)T(ps)C
polyA90ES-FAM
FAM-
(SEQ ID NO: 35)
A(ps)A(ps)A(ps)AAAAAAAAAAAAAAAAAAAAAAAAAAAAAAAAAAAA
AAAAAAAAAAAAAAAAAAAAAAAAAAAAAAAAAAAAAAAAAAAAAAA
A(ps)A(ps)A(ps)A
polyT90ES
FAM-
(SEQ ID NO: 36)
T(ps)T(ps)T(ps)TTTTTTTTTTTTTTTTTTTTTTTTTTTTTTTTTTTT
TTTTTTTTTTTTTTTTTTTTTTTTTTTTTTTTTTTTTTTTTTTTTTT
T(ps)T(ps)T(ps)T

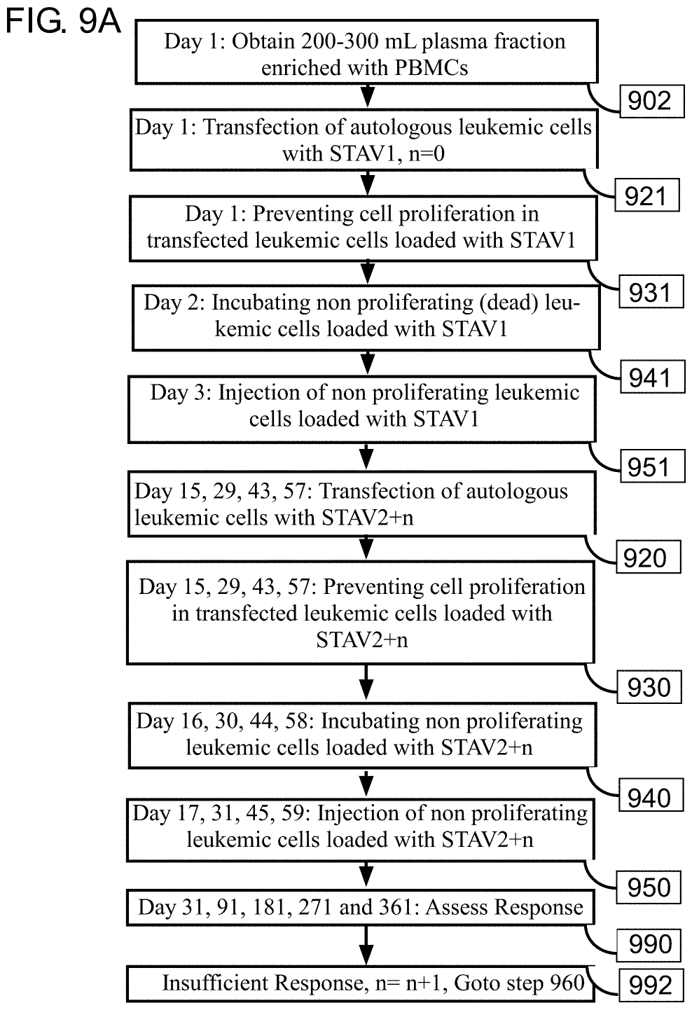

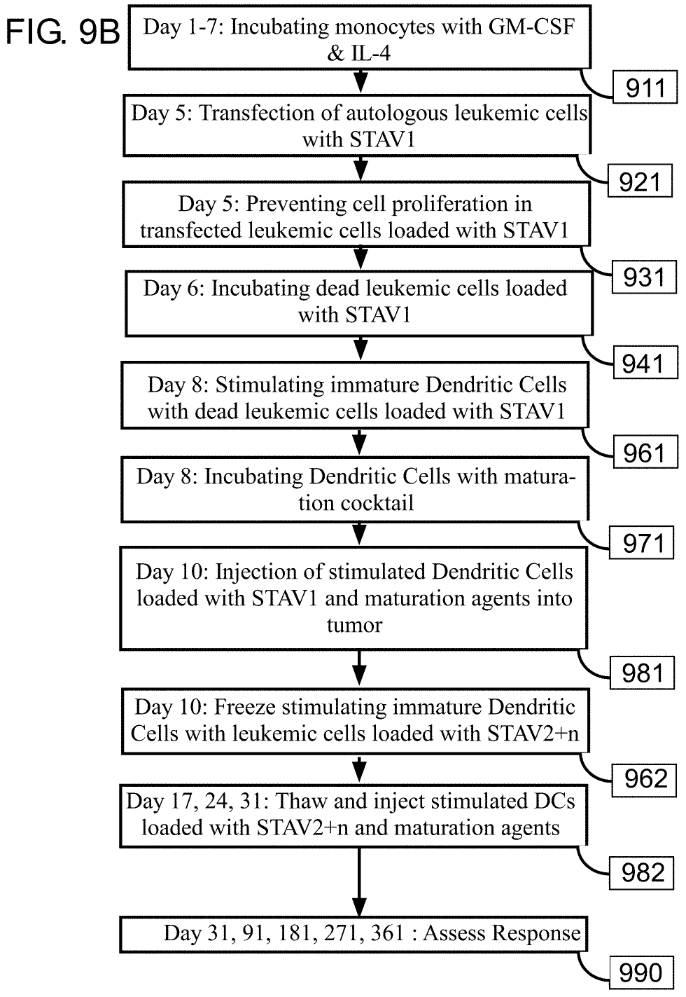

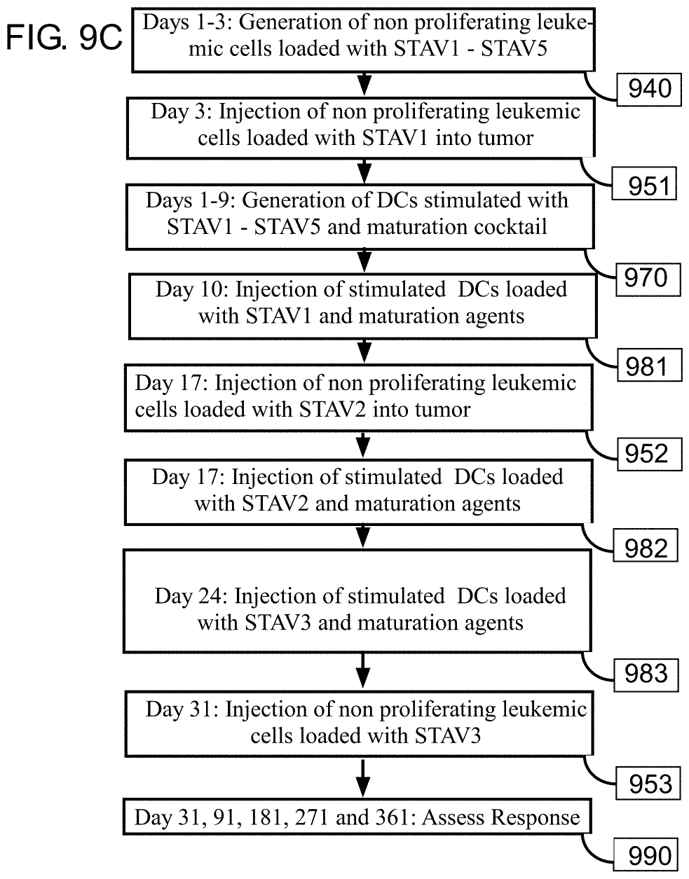

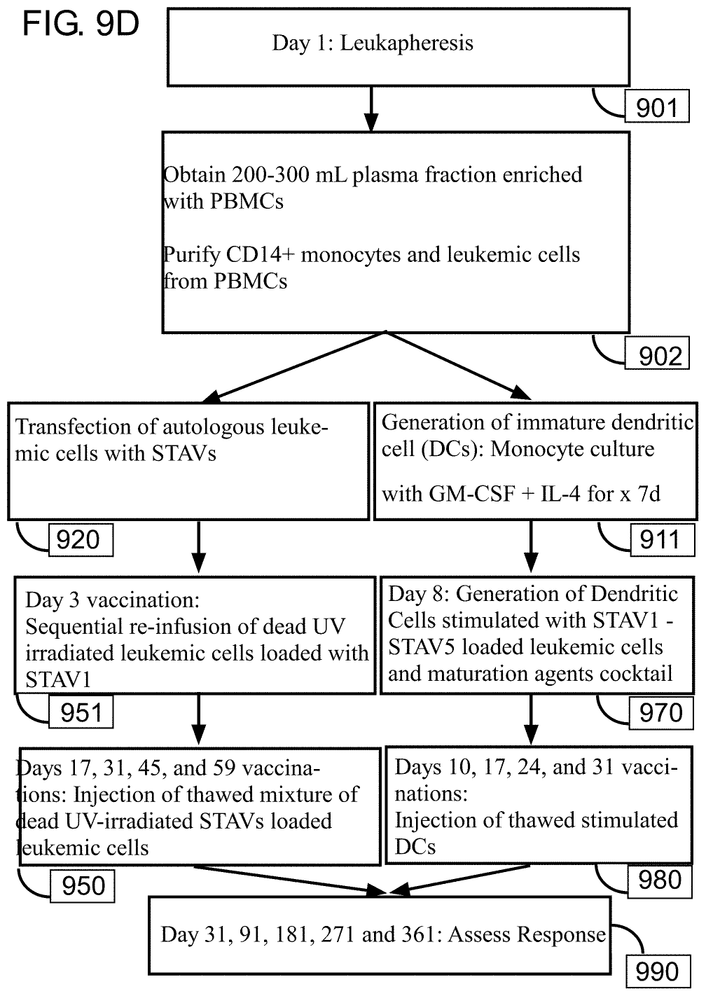

Generation of dendritic cells (DCs): DCs can be generated from monocytes cultured for up to 7 days in the presence of GM-CSF and IL-4, see also FIGS. 9B-9D.

FIG. 9A is a flow diagram showing a treatment protocol for treating a patient with cancer where a plurality of 200-300 mL plasma fraction enriched with PBMCs are obtained at day 1 (902) from the patient's tumor and can be stored at −20° C., where one of the fractions is thawed and transfected with a STAV (e.g., STAV1) (921), where the transfected cells are irradiated with UV light (or otherwise prevented from proliferating) (931) and incubated for a period of time (e.g., 24 h) on day 2 (941) and injected into the tumor on day 3 (951). The procedure is repeated on day 15 with the transfection of a second different STAV, (e.g., STAV2) (920) where the transfected cells are irradiated with UV light (930) and incubated for a period of time (e.g., 24 h) on day 16 (940) and injected into the tumor on day 17 (950). The procedure can be repeated on day 29 with the transfection of a third different STAV, (e.g., STAV3) (920) where the transfected cells are irradiated with UV light (930) and incubated for a period of time (e.g., 24 h) on day 30 (940) and injected into the tumor on day 31 (950). The procedure can be repeated on day 43 with the transfection of a fourth different STAV, (e.g., STAV4) (920) where the transfected cells are irradiated with UV light (930) and incubated for a period of time (e.g., 24 h) on day 44 (940) and injected into the tumor on day 45 (950). The procedure can be repeated on day 57 with the transfection of a fifth different STAV, (e.g., STAVS) (920) where the transfected cells are irradiated with UV light (930) and incubated for a period of time (e.g., 24 h) on day 58 (940) and injected into the tumor on day 59 (950). The response to the treatment can be assessed on day 31, 91, 181, 271 and 361 (990), according to an embodiment of the invention. In an embodiment of the invention, if the response is sufficient the length of time before the next administration of a dead leukemic fraction transfected with a STAV can be extended or delayed.

FIG. 9B is a flow diagram showing a treatment protocol for treating a patient with cancer where a plurality of 200-300 mL plasma fraction enriched with PBMCs are obtained at day 1 from the patient's tumor and can be stored at −20° C. On day 1 through to 7, monocytes are incubated with GM-CSF and IL4 (911). On day 1 one of the leukemic cell fractions is thawed and transfected with a STAV (e.g., STAV1) (921), where the transfected cells are irradiated with UV light (or otherwise prevented from proliferating) (931) and incubated for a period of time (e.g., 24 h) on day 2 (941) and used to stimulate the immature Dendritic Cells on day 8 (961). On day 8 the stimulated Dendritic Cells loaded with the STAV are incubated with maturation agents (971). On day 10 the stimulated Dendritic Cells loaded with the STAV and the maturation agents is injected into the tumor (981). On day 10 stimulated immature Dendritic Cells loaded with a STAV are frozen (e.g., STAV2-STAVS) (962). On day 17, 24, 31 the stimulated Dendritic Cells loaded with a STAV (e.g., STAV2-STAV5) and the maturation agent are thawed and injected into the tumor (982). The response to the treatment can be assessed on day 31, 91, 181, 271 and 361 (990), according to an embodiment of the invention. In an embodiment of the invention, if the response is sufficient the length of time before the next administration of DC's loaded with a STAV can be extended or delayed.

FIG. 9C is a flow diagram showing a treatment protocol including the protocol of treatment shown in FIG. 9A and the protocol of treatment shown in FIG. 9B. On days 1-3, leukemic cells loaded with a STAV are UV irradiated and incubated for a period of time (e.g., 24 h) (940) and either injected into the tumor on day 3 (951) or used to generate the stimulated Dendritic Cells incubated with the maturation cocktail (970). On day 10 the stimulated Dendritic Cells loaded with a STAV and incubated with maturation agents are injected into the tumor (981). On day 17, leukemic cells loaded with a STAV are either injected into the tumor on (952) or used to generate the stimulated Dendritic Cells incubated with the maturation cocktail and are injected into the tumor (982). On day 24 the stimulated Dendritic Cells loaded with a STAV and incubated with maturation agents are injected into the tumor (983). On day 31, leukemic cells loaded with a STAV are injected into the tumor on (953). The response to the treatment can be assessed on day 31, 91, 181, 271 and 361 (990), according to an embodiment of the invention. In an embodiment of the invention, if the response is sufficient the length of time before the next administration of a dead leukemic fraction transfected with a STAV can be extended or delayed.

FIG. 9D is a flow diagram showing a treatment protocol including the protocol of treatment shown in FIG. 9A and the protocol of treatment shown in FIG. 9B treated with up to five STAVs comprising the group consisting of STAV 1, STAV 2, STAV 3, STAV 4 and STAV5. On day 1, the leukemic cells are collected (901). On days 10, 17, 24, and 31 injection of thawed stimulated DCs is carried out (980).

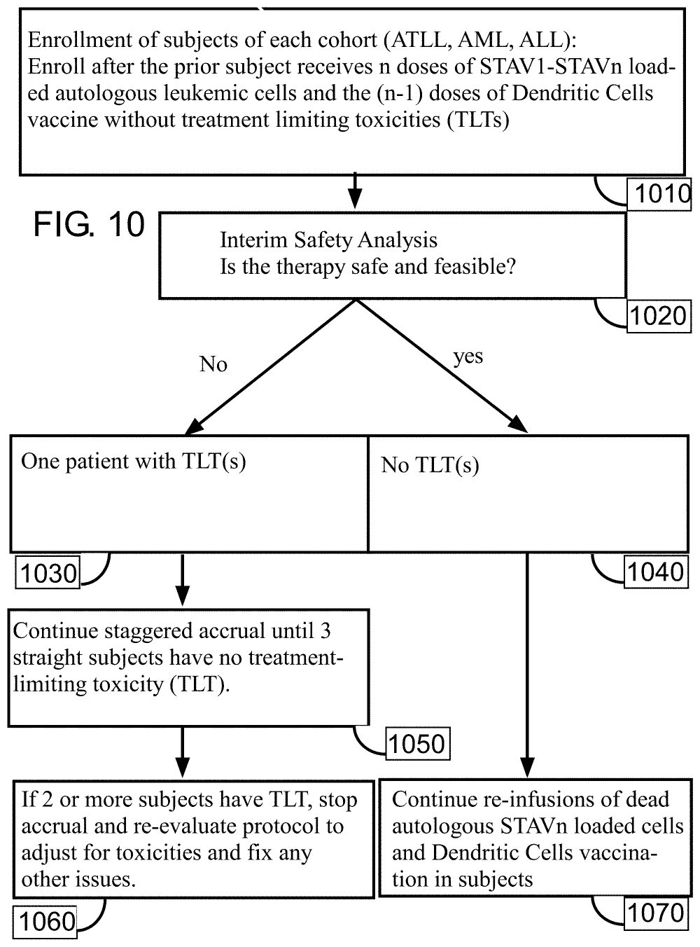

FIG. 10 is a flow diagram showing a limiting toxicity protocol for relapsed/refractory aggressive leukemia. In an embodiment of the present invention, enrollment of subjects of each cohort (ATLL, AML, ALL): enroll after the prior subject receives n doses of STAV1-STAVn loaded autologous leukemic cells and the (n-1) doses of Dendritic Cells vaccine without treatment limiting toxicities (TLTs) (1010). An Interim Safety Analysis is undertaken to ask is the therapy safe and feasible? (1020). If there is one patient with TLT (1030), then there is one patient with TLT (1030), continue staggered accrual until 3 straight subjects have no treatment-limiting toxicity (TLT) (1050). If two or more subjects have TLT, stop accrual and re-evaluate protocol to adjust for toxicities and fix any other issues (1060). If there are no patients with TLT (1040), then continue injections of dead autologous STAVn loaded cells and/or Dendritic Cell vaccinations in subjects (1070).

Days 3, 17, 31, 45, and 59: Sequential, intravenous infusion of fresh or thawed UV irradiated (dead) syngeneic leukemic cells transfected with STAVs 1, 2, 3, 4 and 5 respectively. Days 7-10: In situ DC maturation. Previously cultured immature DCs can be stimulated (loaded) with mixture of thawed STAVs loaded leukemic cells for 24 hours in the presence of maturation agents cocktail consisting of TNF-α and IL-1β for 48-72 hours in order to generate mature DCs days 10, 17, 24, and 31.

Re-infusion of mixture of thawed mature DCs stimulated with leukemic cells previously transfected with STAVs 2, 3, 4, and 5 respectively.

Correlative Studies—Molecular evaluations/analysis in patients with HTLV-1/ATLL: Venous blood can be collected from patients diagnosed with leukemia-type HTLV-1/ATLL at baseline, Day 10, at the ends of Months 1, Month 3, Month, 6, Month, 9, Month 12, an at the end-of-treatment visit after early discontinuation. Collected blood specimens can be processed on the same day (Ramos/Barber labs). PMBCs can be isolated by centrifugation using standard Lymphoprep (ficol) procedure. A portion of fresh or thawed cells can be subjected to magnetic CD4-enrichement by negative selection using commercially available kits. These cells can serve as source for protein and RNA after standard extraction procedures. Non-enriched PBMCs can be used to extract genomic DNA for HTLV-1 pro-viral loads. The extracted cells may be utilized fresh or be cryopreserved in DMSO-liquid nitrogen.

Re-infusion of dead STAVs-loaded HTLV-1/ATLL cells can lead to phagocytosis by APCs in vivo. Such event can result in excess indigestible STAVs that can activate STING dependent signaling within APCs which in turn can facilitate a potent anti-tumor T cell activation. In addition, APCs can present HTLV-1 antigens, such as HBZ (which is always expressed ATLL tumors), which can in turn facilitate CTL priming against HTLV-1 infected cells and eliminate such clones.

CTL assays (STINGINN): To evaluate CTL responses after sequential administrations of STAVs loaded tumor cells and DC vaccinations, venous blood can be collected from patients at baseline, before each DC vaccination on Days 10, 17, 24, 31, 45, and at the end of Months 2, 3, and 6. Collected blood specimens can be processed on the same day. PMBCs can be isolated by centrifugation using standard Lymphoprep (ficol) procedure. The extracted cells may be utilized fresh or be cryopreserved in DMSO-liquid nitrogen.

Methods: HTLV-1 specific CTL responses can be assessed using PBMC isolated from peripheral blood. CD8 T cells can be isolated using human MACS CD8+ T cell isolation kit through negative selection (Miltenyil Biotec, 130-096-495). CD8 T Cells can be plated at 2×105 per well and stimulated with 20 μg/ml of tumor cell lysate protein or overlapping 15-aa peptides covering the envelope, TAX or HBZ region of HTLV-1 for ATLL (custom synthesized by GenScript). After 72 hours stimulation IFN gamma secreting cells can be determined using an ELISPOT assay for human IFNγ and quantitated using a ELISPOT reader system. For flow cytometry, cells can be stimulated for 72 hours. Brefeldin A (3 mg/ml) can be added to the cells 6 h before analysis. Cells can be then washed, stained with cell surface marker (anti-CD3, anti-CD8), permeabilized with Cytofix/Cytoperm (BD Biosciences), and stained with IFNγ. Data can be acquired using an LSR II flow cytometer.

The generation of tumor antigen specific T cells constitutes an important host defense response that evolved in part to eliminate the development of cancer. The mechanisms underlining the stimulation of antigen presenting cells (APCs) and the priming of tumor-specific T cells are still unclear but implicate the generation of immune stimulatory type I interferon and other cytokines triggered by engulfed dead cells. The stimulation of innate immune signaling pathways leading to cytokine production within phagocytes such as CD8+ dendritic cells (DCs) involves the innate immune sensor STING. STING directly senses cyclic dinucleotides (CDNs) including c-di-GMP or c-di-AMP secreted by invading intracellular bacteria or c-GMP-AMP generated by cGAS, and cytosolic dsDNA species such as microbial DNA or even self-DNA. Generally, the cytosol is free of DNA as it would aggravate endogenous STING-dependent cytokine production, an event that can lead to lethal inflammation and autoimmunity. Self-DNA leaked from the nucleus, following cell division or DNA damage is prevented from activating STING signaling by the exonuclease Trex1 (DNase III). In addition, following the engulfment of apoptotic cells, phagocyte-dependent DNase II plays a critical role in digesting DNA within dead cells thus preventing it from activating STING-signaling. Further, loss of DNase II function has been shown to be embryonic lethal in murine models due to high-level cytokine production being instigated by overactive STING activity.

Cytosolic STING activators, including dsDNA are usually only generated by microbial infection or following DNA-damage events and can render tumor cells highly immunogenic. STINGINN has developed DNase-resistant nucleic acid-based STING-dependent innate immune agonists, referred to as STAVs (e.g., dsDNA species of <80 nucleotides in length). Autologous tumor cells are loaded with STAVs and irradiated. After in vitro or in vivo phagocytosis, the DNase-resistant STAVs activate STING signaling in the APC, in trans, which facilitates the cross-presentation of tumor antigen and the priming of anti-tumor T cells (makes a ‘cold’ tumor ‘hot’). This strategy can be applicable to multiple tumor types.

Abbreviations used in the following examples and elsewhere herein are: AcOH acetic acid; ALL Adult Lymphocytic Leukemia; AML Acute Myeloid Leukemia; ANA anti nuclear antibody; APC Antigen Presenting Cells; ATLL Adult T cell Lymphocytic Leukemia; ATM atmosphere; BMDM bone marrow derived macrophages; BOC₂O di-tert-butyl dicarbonate; cGAMP cyclic [G(2′,5′)pA(3′,5′)p]; cGAS

cyclic guano sine monophosphate-adenosine monophosphate synthase; CuSO₄ copper sulfate; CDCl₃ deuterated chloroform; CDN cyclic dinucleotides; CTL Cytotoxic T cells; DC dendritic cells; DCM dichloromethane; DIEA N,N-diisopropylethylamine; DMA N,N-dimethylacetamide; DMAP 4-dimethylaminopyridine; DMF N,N-dimethylformamide; DMSO dimethyl sulfoxide; DMSO-d₆ deuterated dimethyl sulfoxide; dsDNA double stranded deoxyribonucleic acid; EDCI 1-ethyl-3-(3-dimethylaminopropyl) carbodiimide; ER Endoplasmic Reticulum; ESI electro spray ionization; EtOAc ethyl acetate; FAM fluorescein; H&E hematoxylin and eosin; HCl hydrochloric acid; h

hour(s); HPLC high-performance liquid chromatography; hTERT immortalized human fibroblasts; HTLV-1 Human T-cell leukemia/lymphoma virus type 1; IFN interferon; IRF3 Interferon Regulatory Factor 3; IRF7 Interferon Regulatory Factor 7; ISD

Interferon Stimulatory DNA; i.t. intratumorally; LCMS liquid chromatography—mass spectrometry; MDA5 Melanoma Differentiation associated Antigen 5; mL milliliter; MeCN

acetonitrile; MEF Murine Embryonic Fibroblasts; MeOH methanol; mg milligram; mmolmillimole; MgSO₄ magnesium sulfate; MHz megahertz; min minutes; MS

mass spectrometry; MEF Murine Embryonic Fibroblasts; Na₂CO₃ sodium carbonate; NaHCO₃ sodium bicarbonate; NF-kB nuclear factor kappa-light-chain-enhancer of activated B cells; NMR nuclear magnetic resonance; PCR polymerase chain reaction; s.c.

subcutaneously; SEAP secreted alkaline phosphatase; STAV STING-dependent adjuvants; STING Stimulator of Interferon Genes; Tf triflate; TKO TREX1 KnockOut; TM transmembrane; Pd₂(dba)₃ tris(dibenzylideneacetone)dipalladium(0); Pd(PPh₃)₂Cl₂ bis(triphenylphosphine)palladium(II) dichloride; PAMP pathogen associated molecular patterns; ppm parts per million; polyIC Polyinosinic:polycytidylic acid; PCR polymerase chain reaction; PTSA para-toluene sulfonic acid; qPCR quantitative real time PCR; RIG-1 Retinoic acid Inducible Gene 1; RNA Ribonucleic Acid; RT room temperature; t-BuOH tert-butanol; TBAF tetra-n-butylammonium fluoride; TBK1

TANK-binding kinase 1; THF tetrahydrofuran; TRAP translocon-associated protein; TFA trifluoroacetic acid; TLR Toll-like receptors; TMS trimethylsilane; TLC thin layer chromatography; TSA thermal shift assay; μL microliter; UV ultraviolet; VSV

vesicular stomatitis virus; WT Wild Type.

In an embodiment of the present invention, autologous tumor cells loaded with STING-dependent adjuvants can be reinfused into a patient to stimulate APCs in vivo and thus anti-tumor CTL's. STAV loaded cells are highly immunogenic, and therefore potent activators of APC's. In various embodiments of the present invention, the strategy is applicable to patients suffering from a highly aggressive leukemia, e.g., relapsed/refractory acute myeloid leukemia (AML) or adult lymphocytic leukemia (ALL) such as HTLV-1 associated adult T cell lymphocytic leukemia (ATLL). In various alternative embodiments of the present invention, the strategy is applicable to a variety of cancers, not just leukemia. Data demonstrate that UV irradiated (dead) AML, ALL, and ATLL cells preloaded with STAVs potently induced STING signaling in human macrophages in trans, following phagocytosis. Preclinical animal data also indicates that irradiated EL4 or C1498 (murine ALL models) cells loaded with STAVs potently activates APCs in trans, in a STING-dependent manner, in vivo, to generate anti-tumor T cells. This treatment can protect cancer carrying mice from tumor development. No toxic effects were observed in normal blood circulating myeloid and lymphoid cell lineages, supporting the potential safety of a personalized approach using serially injected irradiated autologous STAVs-loaded leukemic cells. Further embodiments.

Embodiments contemplated herein include Embodiments P1-P42 following.

Embodiment P1. A method for treating a human subject suffering from cancer including isolating a plurality of tumor cells from a human subject having cancer, generating a plurality of first incubated tumor cells including the steps of transfecting a first STing dependent ActiVator (STAV) to a plurality of tumor cells to generate a plurality of first transfected tumor cells, where the first STAV is selected from the group consisting of STAV 1, STAV 2, STAV 3, STAV 4 and STAV5, treating the plurality of first transfected tumor cells to prevent cell proliferation to generate a plurality of first dead tumor cells, and incubating the plurality of first dead tumor cells to generate a plurality of first incubated tumor cells, infusing the plurality of first incubated tumor cells into the human subject, generating a plurality of second incubated tumor cells including the steps of transfecting a second STAV to a plurality of tumor cells to generate a plurality of second transfected tumor cells, where the second STAV is selected from the group consisting of STAV 1, STAV 2, STAV 3, STAV 4 and STAV 5, provided however that the second STAV is not the same as the first STAV, treating the plurality of second transfected tumor cells to prevent cell proliferation to generate a plurality of second dead tumor cells, and incubating the plurality of second dead tumor cells to generate a plurality of second incubated tumor cells; and infusing the plurality of second incubated tumor cells into the human subject, thereby treating the human subject suffering from cancer.

Embodiment P2. The method of Embodiment P1, where the step of treating to prevent proliferation of the transfected tumor cells utilizes ultraviolet (UV) light.

Embodiment P3. The method of Embodiment P2, where the transfected cells are exposed to UV light for between a lower limit of approximately ten (10) mJoule; and an upper limit of approximately one (1) Joule.

Embodiment P4. The method of Embodiment P1, where the step of treating to prevent proliferation of the transfected tumor cells utilizes x-rays to irradiate the cells.

Embodiment P5. The method of Embodiment P1, where the time period elapsed between infusing the plurality of first incubated tumor cells and infusing the plurality of second incubated tumor cells is between a lower limit of approximately two (2) days and an upper limit of approximately twenty (20) days.

Embodiment P6. The method of Embodiment P1, where the time period elapsed between treating the plurality of first transfected tumor cells and infusing the plurality of first incubated tumor cells is between a lower limit of approximately two (2) hours, and an upper limit of approximately five (5) days.

Embodiment P7. The method of Embodiment P1, where the STAV is transfected for between: a lower limit of approximately one (1) hour; and an upper limit of approximately five (5) hours.

Embodiment P8. A method for treating a human subject suffering from cancer including isolating a plurality of tumor cells and a plurality of monocyte cells from a human subject having cancer, generating a plurality of first incubated tumor cells including the steps of transfecting a first STing dependent ActiVator (STAV) to a plurality of tumor cells to generate a plurality of first transfected tumor cells, where the first STAV is selected from the group consisting of STAV 1, STAV 2, STAV 3, STAV 4 and STAV 5, treating the plurality of first transfected tumor cells to prevent cell proliferation to generate a plurality of first dead tumor cells and incubating the plurality of first dead tumor cells to generate a plurality of first incubated tumor cells, infusing the plurality of first incubated tumor cells into the human subject, generating a plurality of first stimulated Dendritic Cells (DCs) including the steps of incubating a plurality of monocyte cells with one or both GM-CSF and IL-4 to generate a plurality of first immature DCs, stimulating the plurality of first immature DCs with the plurality of first incubated tumor cells to generate a plurality of first leukemic stimulated DCs, and incubating the plurality of first leukemic stimulated DCs with a maturation cocktail to generate a plurality of first stimulated DCs, infusing the plurality of first stimulated DC's into the human subject, thereby treating the human subject suffering from cancer

Embodiment P9. The method of Embodiment P8, where the step of treating to prevent proliferation of the transfected tumor cells utilizes ultraviolet (UV) light.

Embodiment P10. The method of Embodiment P9, where the transfected cells are exposed to UV light for between a lower limit of approximately ten (10) mJoule; and an upper limit of approximately one (1) Joule.

Embodiment P11. The method of Embodiment P8, where the step of treating to prevent proliferation of the transfected tumor cells utilizes x-rays to irradiate the cells.

Embodiment P12. The method of Embodiment P8, where the time period elapsed between infusing the plurality of first incubated tumor cells and infusing the plurality of second incubated tumor cells is between a lower limit of approximately two (2) days and an upper limit of approximately twenty (20) days.

Embodiment P13. The method of Embodiment P8, where the time period elapsed between treating the plurality of first transfected tumor cells and infusing the plurality of first incubated tumor cells is between a lower limit of approximately two (2) hours, and an upper limit of approximately five (5) days.

Embodiment P14. The method of Embodiment P8, where the STAV is transfected for between: a lower limit of approximately one (1) hour; and an upper limit of approximately five (5) hours.

Embodiment P15. A method for treating a human subject suffering from cancer including isolating a plurality of tumor cells and a plurality of monocyte cells from a human subject having cancer, generating a plurality of first incubated tumor cells including the steps of transfecting a first STing dependent ActiVator (STAV) to a plurality of tumor cells to generate a plurality of first transfected tumor cells, where the first STAV is selected from the group consisting of STAV 1, STAV 2, STAV 3, STAV 4 and STAV 5, treating the plurality of first transfected tumor cells to prevent cell proliferation to generate a plurality of first dead tumor cells, and incubating the plurality of first dead tumor cells to generate a plurality of first incubated tumor cells, infusing the plurality of first incubated tumor cells into the human subject, generating a plurality of first stimulated Dendritic Cells (DCs) including the steps of incubating a plurality of monocyte cells with one or both GM-CSF and IL-4 to generate a plurality of first immature DCs, stimulating the plurality of first immature DCs with the plurality of first incubated tumor cells to generate a plurality of first leukemic stimulated DCs and incubating the plurality of first leukemic stimulated DCs with a maturation cocktail to generate a plurality of first stimulated DCs, infusing the plurality of first stimulated DC's into the human subject, generating a plurality of second incubated tumor cells including the steps of transfecting a second STAV to a plurality of tumor cells to generate a plurality of second transfected tumor cells, where the second STAV is selected from the group consisting of STAV 1, STAV 2, STAV 3, STAV 4 and STAV 5, provided however that the second STAV is not the same as the first STAV, treating the plurality of second transfected tumor cells to prevent cell proliferation to generate a plurality of second dead tumor cells and incubating the plurality of second dead tumor cells to generate a plurality of second incubated tumor cells and infusing the plurality of second incubated tumor cells into the human subject, thereby treating the human subject suffering from cancer.

Embodiment P16. The method of Embodiment P15, where the step of treating to prevent proliferation of the transfected tumor cells utilizes ultraviolet (UV) light.

Embodiment P17. The method of Embodiment P16, where the transfected cells are exposed to UV light for between a lower limit of approximately ten (10) mJoule; and an upper limit of approximately one (1) Joule.

Embodiment P18. The method of Embodiment P15, where the step of treating to prevent proliferation of the transfected tumor cells utilizes x-rays to irradiate the cells.

Embodiment P19. The method of Embodiment P15, where the time period elapsed between infusing the plurality of first incubated tumor cells and infusing the plurality of second incubated tumor cells is between a lower limit of approximately two (2) days and an upper limit of approximately twenty (20) days.

Embodiment P20. The method of Embodiment P15, where the time period elapsed between treating the plurality of first transfected tumor cells and infusing the plurality of first incubated tumor cells is between a lower limit of approximately two (2) hours, and an upper limit of approximately five (5) days.

Embodiment P21. The method of Embodiment P15, where the STAV is transfected for between: a lower limit of approximately one (1) hour; and an upper limit of approximately five (5) hours.

Embodiment P22. The method of Embodiment P15, where the maturation cocktail comprises incubation for between a minimum of twenty four (24) hours and a maximum of ninety six (96) hours with TNF-α and IL-1β.

Embodiment P22. A method for treating a human subject suffering from cancer including isolating a plurality of tumor cells and a plurality of monocyte cells from a human subject having cancer, generating a plurality of first incubated tumor cells including the steps of transfecting a first STing dependent ActiVator (STAV) to a plurality of tumor cells to generate a plurality of first transfected tumor cells, where the first STAV is selected from the group consisting of STAV 1, STAV 2, STAV 3, STAV 4 and STAV 5, treating the plurality of first transfected tumor cells to prevent cell proliferation to generate a plurality of first dead tumor cells, and incubating the plurality of first dead tumor cells to generate a plurality of first incubated tumor cells, infusing the plurality of first incubated tumor cells into the human subject, generating a plurality of first stimulated Dendritic Cells (DCs) including the steps of incubating a plurality of monocyte cells with one or both GM-CSF and IL-4 to generate a plurality of first immature DCs, stimulating the plurality of first immature DCs with the plurality of first incubated tumor cells to generate a plurality of first leukemic stimulated DCs, and incubating the plurality of first leukemic stimulated DCs with a maturation cocktail to generate a plurality of first stimulated DCs, infusing the plurality of first stimulated DC's into the human subject, generating a plurality of second stimulated DCs including the steps of stimulating a plurality of first immature DCs with the plurality of second incubated tumor cells to generate a plurality of second leukemic stimulated DCs, and incubating the plurality of second leukemic stimulated DCs with a maturation cocktail to generate a plurality of second stimulated DCs; and g) infusing the plurality of second stimulated DC's into the human subject, thereby treating the human subject suffering from cancer.

Embodiment P23. The method of Embodiment P22, where the step of treating to prevent proliferation of the transfected tumor cells utilizes ultraviolet (UV) light.

Embodiment P24. The method of Embodiment P23, where the transfected cells are exposed to UV light for between a lower limit of approximately ten (10) mJoule; and an upper limit of approximately one (1) Joule.

Embodiment P25. The method of Embodiment P22, where the step of treating to prevent proliferation of the transfected tumor cells utilizes x-rays to irradiate the cells.

Embodiment P26. The method of Embodiment P22, where the time period elapsed between infusing the plurality of first incubated tumor cells and infusing the plurality of second incubated tumor cells is between a lower limit of approximately two (2) days and an upper limit of approximately twenty (20) days.

Embodiment P27. The method of Embodiment P22, where the time period elapsed between treating the plurality of first transfected tumor cells and infusing the plurality of first incubated tumor cells is between a lower limit of approximately two (2) hours, and an upper limit of approximately five (5) days.

Embodiment P28. The method of Embodiment P22, where the STAV is transfected for between: a lower limit of approximately one (1) hour; and an upper limit of approximately five (5) hours.

Embodiment P29. The method of Embodiment P22, where the maturation cocktail comprises incubation for between a minimum of twenty four (24) hours and a maximum of ninety six (96) hours with TNF-α and IL-1β.

Embodiment P30. A method for treating a human subject suffering from cancer including isolating a plurality of tumor cells and a plurality of monocyte cells from a human subject having cancer, generating a plurality of first stimulated Dendritic Cells (DCs) including the steps of transfecting a first STing dependent ActiVator (STAV) to a plurality of tumor cells to generate a plurality of first transfected tumor cells, where the first STAV is selected from the group consisting of STAV 1, STAV 2, STAV 3, STAV 4 and STAV 5, treating the plurality of first transfected tumor cells to prevent cell proliferation to generate a plurality of first dead tumor cells, and incubating the plurality of first dead tumor cells to generate a plurality of first incubated tumor cells, incubating a plurality of monocyte cells with one or both GM-CSF and IL-4 to generate a plurality of first immature DCs, stimulating the plurality of first immature DCs with the plurality of first incubated tumor cells to generate a plurality of first leukemic stimulated DCs, and incubating the plurality of first leukemic stimulated DCs with a maturation cocktail to generate a plurality of first stimulated DCs, infusing the plurality of first stimulated DC's into the human subject, generating a plurality of second stimulated DCs including the steps of transfecting a second STAV to a plurality of tumor cells to generate a plurality of second transfected tumor cells, where the second STAV is selected from the group consisting of STAV 1, STAV 2, STAV 3, STAV 4 and STAV 5, provided however that the second STAV is not the same as the first STAV, treating the plurality of second transfected tumor cells to prevent cell proliferation to generate a plurality of second dead tumor cells, and incubating the plurality of second dead tumor cells to generate a plurality of second incubated tumor cells and stimulating a plurality of first immature DCs with the plurality of second incubated tumor cells to generate a plurality of second leukemic stimulated DCs and incubating the plurality of second leukemic stimulated DCs with a maturation cocktail to generate a plurality of second stimulated DCs and infusing the plurality of second stimulated DC's into the human subject, thereby treating the human subject suffering from cancer.

Embodiment P31. The method of Embodiment P30, where the step of treating to prevent proliferation of the transfected tumor cells utilizes ultraviolet (UV) light.

Embodiment P32. The method of Embodiment P31, where the transfected cells are exposed to UV light for between a lower limit of approximately ten (10) mJoule; and an upper limit of approximately one (1) Joule.

Embodiment P33. The method of Embodiment P30, where the step of treating to prevent proliferation of the transfected tumor cells utilizes x-rays to irradiate the cells.

Embodiment P34. The method of Embodiment P30, where the time period elapsed between infusing the plurality of first incubated tumor cells and infusing the plurality of second incubated tumor cells is between a lower limit of approximately two (2) days and an upper limit of approximately twenty (20) days.

Embodiment P35. The method of Embodiment P30, where the time period elapsed between treating the plurality of first transfected tumor cells and infusing the plurality of first incubated tumor cells is between a lower limit of approximately two (2) hours, and an upper limit of approximately five (5) days.

Embodiment P36. The method of Embodiment P30, where the STAV is transfected for between: a lower limit of approximately one (1) hour; and an upper limit of approximately five (5) hours.

Embodiment P37. The method of Embodiment P30, where the maturation cocktail comprises incubation for between a minimum of twenty four (24) hours and a maximum of ninety six (96) hours with TNF-α and IL-1β.

Embodiment P38. A method for treating a human subject suffering from cancer including isolating a plurality of tumor cells and a plurality of monocyte cells from a human subject having cancer, generating a plurality of first incubated tumor cells including the steps of transfecting a first STing dependent ActiVator (STAV) to a plurality of tumor cells to generate a plurality of first transfected tumor cells, where the first STAV is selected from the group consisting of STAV 1, STAV 2, STAV 3, STAV 4 and STAV 5, treating the plurality of first transfected tumor cells to prevent cell proliferation to generate a plurality of first dead tumor cells and incubating the plurality of first dead tumor cells to generate a plurality of first incubated tumor cells, infusing the plurality of first incubated tumor cells into the human subject, generating a plurality of first stimulated Dendritic Cells (DCs) including the steps of incubating a plurality of monocyte cells with one or both GM-CSF and IL-4 to generate a plurality of first immature DCs, stimulating the plurality of first immature DCs with the plurality of first incubated tumor cells to generate a plurality of first leukemic stimulated DCs, and incubating the plurality of first leukemic stimulated DCs with a maturation cocktail to generate a plurality of first stimulated DCs, infusing the plurality of first stimulated DC's into the human subject, generating a plurality of second incubated tumor cells including the steps of transfecting a second STAV to a plurality of tumor cells to generate a plurality of second transfected tumor cells, where the second STAV is selected from the group consisting of STAV 1, STAV 2, STAV 3, STAV 4 and STAV 5, provided however that the second STAV is not the same as the first STAV, treating the plurality of second transfected tumor cells to prevent cell proliferation to generate a plurality of second dead tumor cells and incubating the plurality of second dead tumor cells to generate a plurality of second incubated tumor cells and infusing the plurality of second incubated tumor cells into the human subject, generating a plurality of second stimulated DCs including the steps of stimulating a plurality of first immature DCs with the plurality of second incubated tumor cells to generate a plurality of second leukemic stimulated DCs, and incubating the plurality of second leukemic stimulated DCs with a maturation cocktail to generate a plurality of second stimulated DCs and infusing the plurality of second stimulated DC's into the human subject, thereby treating the human subject suffering from cancer.

Embodiment P39. A composition for treating a human subject suffering from cancer including a first double-stranded DNA STing dependent ActiVator (STAV) including a first single-stranded DNA selected from the group consisting of single-stranded Poly A76ES, single-stranded Poly AC76ES, single-stranded Poly AT76ES, single-stranded Poly ACT76ES, single-stranded HSV RL2 intron-S, and a second single-stranded DNA, where when the first single-stranded DNA is single-stranded Poly A76ES, then the second single-stranded DNA is single-stranded Poly T76ES, where when the first single-stranded DNA is single-stranded Poly AC76ES, then the second single-stranded DNA is single-stranded Poly TG76ES, where when the first single-stranded DNA is single-stranded Poly AT76ES, then the second single-stranded DNA is single-stranded Poly AT76ES, where when the first single-stranded DNA is single-stranded Poly ACT76ES, then the second single-stranded DNA is single-stranded Poly CAG76ES, where when the first single-stranded DNA is single-stranded HSV RL2 intron-S, then the second single-stranded DNA is single-stranded HSV RL2 intron-AS and a second double-stranded DNA STAV including a third single-stranded DNA selected from the group consisting of single-stranded Poly A76ES, single-stranded Poly AC76ES, single-stranded Poly AT76ES, single-stranded Poly ACT76ES, single-stranded HSV RL2 intron-S, where the third single-stranded DNA is not the same as the first single-stranded DNA and a fourth single-stranded DNA, where when the third single-stranded DNA is single-stranded Poly A76ES, then the fourth single-stranded DNA is single-stranded Poly T76ES, where when the third single-stranded DNA is single-stranded Poly AC76ES, then the fourth single-stranded DNA is single-stranded Poly TG76ES, where when the third single-stranded DNA is single-stranded Poly AT76ES, then the fourth single-stranded DNA is single-stranded Poly AT76ES, where when the third single-stranded DNA is single-stranded Poly ACT76ES, then the fourth single-stranded DNA is single-stranded Poly CAG76ES, where when the third single-stranded DNA is single-stranded HSV RL2 intron-S, then the fourth single-stranded DNA is single-stranded HSV RL2 intron-AS.

Embodiment P40. A kit for treating a human subject suffering from cancer including a double-stranded DNA STing dependent ActiVator (STAV) with sequence corresponding to STAV 1, a double-stranded DNA STAV with sequence corresponding to STAV 2, a double-stranded DNA STAV with sequence corresponding to STAV 3, a double-stranded DNA STAV with sequence corresponding to STAV 4, a double-stranded DNA STAV with sequence corresponding to STAV5, and instructions for (i) collecting Peripheral Blood Mononuclear Cells (PBMCs), transfecting the PBMCs with one or more STAVs to generate transfected tumor cells, treating the transfected tumor cells to prevent cell proliferation, incubating the dead tumor cells and infusing the incubated dead tumor cells into the human subject.

Embodiment P41. A kit for treating a human subject suffering from cancer including three or more of STAVs selected from the group consisting of a) a double-stranded DNA including a single-stranded Poly A76ES and a single-stranded Poly T76ES, a double-stranded DNA including a single-stranded Poly AC76ES and a single-stranded Poly TG76ES, a double-stranded DNA including a single-stranded Poly AT76ES and a single-stranded Poly AT76ES, a double-stranded DNA including a single-stranded Poly ACT76ES and a single-stranded Poly CAG76ES; and e) a double-stranded DNA including a single-stranded HSV RL2 intron-S and a single-stranded HSV RL2 intron-AS; and instructions including directions for collecting Peripheral Blood Mononuclear Cells (PBMCs), directions for transfecting the PBMCs with two or more STAVs to generate transfected tumor cells, directions for treating the transfected tumor cells to prevent cell proliferation, directions for incubating the dead tumor cells, and directions for infusing the incubated dead tumor cells into the human subject.

Embodiment P42. A method for treating a human subject suffering from cancer including isolating a plurality of tumor cells from a human subject having cancer, generating a plurality of first incubated tumor cells including a step for transfecting a first STing dependent ActiVator (STAV) to a plurality of tumor cells to generate a plurality of first transfected tumor cells, a step for treating the plurality of first transfected tumor cells to prevent cell proliferation to generate a plurality of first dead tumor cells; and a step for incubating the plurality of first dead tumor cells to generate a plurality of first incubated tumor cells, infusing the plurality of first incubated tumor cells into the human subject, generating a plurality of second incubated tumor cells including the steps of a step for transfecting a second STAV to a plurality of tumor cells to generate a plurality of second transfected tumor cells, provided however that the second STAV is not the same as the first STAV, a step for treating the plurality of second transfected tumor cells to prevent cell proliferation to generate a plurality of second dead tumor cells and a step for incubating the plurality of second dead tumor cells to generate a plurality of second incubated tumor cells, and infusing the plurality of second incubated tumor cells into the human subject, thereby treating the human subject suffering from cancer.

### EQUIVALENTS

Those skilled in the art will recognize, or be able to ascertain using no more than routine experimentation, many equivalents to the specific embodiments and methods described herein. Such equivalents are intended to be encompassed by the scope of the present application. All patents, patent applications, and literature references cited herein are hereby expressly incorporated by reference.

Example embodiments of the methods, systems, and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. For example, it is envisaged that, irrespective of the actual shape depicted in the various Figures and embodiments described above, the outer diameter exit of the inlet tube can be tapered or non-tapered and the outer diameter entrance of the outlet tube can be tapered or non-tapered.

Thus, the breadth and scope of the present invention should not be limited by any of the above-described exemplary embodiments, but should be defined only in accordance with the following claims and their equivalents.

What is claimed is:

1. A method for mediating a DNA induced innate immune response in a subject having a disease associated with aberrant STING function, the method comprising:
a) administering a primary immunization of the subject with a first pharmaceutical composition comprising a first double-stranded DNA vector which modulates STING function and a pharmaceutically acceptable carrier; and
b) administering a booster immunization after the primary immunization with a second pharmaceutical composition comprising a second double-stranded DNA vector which modulates STING function and a pharmaceutically acceptable carrier, where the method is effective to ameliorate the aberrant STING function in the subject.

2. The method of claim 1, where the booster immunization is given between:
a lower limit of approximately 2 weeks after the primary immunization; and
an upper limit of approximately 4 weeks after the primary immunization.

3. The method of claim 1, where one or both the first double-stranded DNA vector and the second double-stranded DNA vector is between 80 base pairs in length and 100 base pairs in length.

4. The method of claim 3, where one or both the first double-stranded DNA vector and the second double-stranded DNA vector comprises two or more nuclease-resistant nucleotides.

5. The method of claim 3, where the first pharmaceutical composition is a double-stranded DNA vector selected from the group consisting of poly dA-dT, poly dC-dG and interferon stimulating DNA (ISD).

6. The method of claim 5, where the first double-stranded DNA vector comprises two or more nuclease-resistant nucleotides.

7. The method of claim 5, where the second pharmaceutical composition is a double-stranded DNA vector selected from the group consisting of poly dA-dT, poly dC-dG and ISD.

8. The method of claim 7, where the second double-stranded DNA vector comprises two or more nuclease-resistant nucleotides.

9. The method of claim 1, where the disease or disorder is a DNA-dependent inflammatory disease.

10. The method of claim 1, where one or both the primary immunization and the secondary immunization is through intramuscular electroporation.

11. A method for stimulating an immune response in a human subject, where the human subject is suffering from a disease associated with aberrant STING function, the method comprising:
a) determining whether the human subject is suffering from a disease associated with aberrant STING function;
b) if the human subject is suffering from a disease associated with aberrant STING function, then administering a primary immunization of the subject with a first pharmaceutical composition comprising a first double-stranded DNA vector which modulates STING function and a pharmaceutically acceptable carrier; and
c) administering a booster immunization after the primary immunization with a second pharmaceutical composition comprising a second double-stranded DNA vector which modulates STING function and a pharmaceutically acceptable carrier, where the method is effective to ameliorate the aberrant STING function in the subject.

12. The method of claim 11, where the booster immunization is given between:
a lower limit of approximately 2 weeks after the primary immunization; and
an upper limit of approximately 4 weeks after the primary immunization.

13. The method of claim 11, where one or both the first double-stranded DNA vector and the second double-stranded DNA vector is between 80 base pairs in length and 100 base pairs in length.

14. The method of claim 13, where one or both the first double-stranded DNA vector and the second double-stranded DNA vector comprises two or more nuclease-resistant nucleotides.

15. The method of claim 13, where the first pharmaceutical composition is a double-stranded DNA vector selected from the group consisting of poly dA-dT, poly dC-dG and interferon stimulating DNA (ISD).

16. The method of claim 15, where the first double-stranded DNA vector comprises two or more nuclease-resistant nucleotides.

17. The method of claim 15, where the second pharmaceutical composition is a double-stranded DNA vector selected from the group consisting of poly dA-dT, poly dC-dG and ISD.

18. The method of claim 17, where the second double-stranded DNA vector comprises two or more nuclease-resistant nucleotides.

19. The method of claim 11, where the disease or disorder is a DNA-dependent inflammatory disease.

20. The method of claim 11, where one or both the primary immunization and the secondary immunization is through intramuscular electroporation.
